(19) United States
(12) Patent Application Publication
(10) Pub. No.: US 2024/0358766 A1
(43) Pub. Date: Oct. 31, 2024

(54) ALLOGENEIC CULTURED KERATINOCYTE PRODUCTS

(71) Applicant: Stratatech Corporation, Madison, WI (US)

(72) Inventors: Allen R. Comer, Madison, WI (US); Kenneth R. Gratz, Madison, WI (US)

(21) Appl. No.: 18/565,007

(22) PCT Filed: May 27, 2022
(86) PCT No.: PCT/US2022/031402

§ 371 Date: Nov. 28, 2023

Related U.S. Application Data
(60) Provisional application No. 63/194,672, filed on May 28, 2021.
(60) Provisional application No. 63/224,706, filed on Jul. 22, 2021.
(60) Provisional application No. 63/227,119, filed on Jul. 29, 2021.

(51) Int. Cl. A61K 35/36; A61F 2/10; A61L 27/24; A61L 27/38; A61L 27/60; A61P 17/02

(52) U.S. Cl. CPC A61K 35/36; A61F 2/105; A61L 27/24; A61L 27/3813; A61L 27/60; A61P 17/02; A61L 2430/34

(57) ABSTRACT

The present disclosure is directed to allogeneic cultured keratinocyte products.

### FIELD OF THE DISCLOSURE

The present disclosure is directed to allogeneic cultured keratinocyte products.

### BACKGROUND

Patients who suffer severe thermal burns face a long and painful recovery. When surgical intervention is required, such as in some deep partial-thickness burns, a patient's own skin or skin from a cadaver may be grafted to the wounded area. Although autografts reduce the chance of rejection that comes with use of cadaver skin, it requires the patient to endure further trauma. What is needed are grafts that promote healing and have a low chance of being rejected by a patient.

### SUMMARY OF THE DISCLOSURE

Among the various aspects of the disclosure may be a composition of allogeneic cultured keratinocytes for topical use, the composition comprising an allogeneic cellularized scaffold, wherein the scaffold further comprises dermal fibroblasts and murine collagen.

The keratinocytes and dermal fibroblasts are karyo-typically stable. The keratinocytes and dermal fibroblasts do not exhibit anchorage-independent growth. The composition comprises no detectable pathogens. The composition secretes human growth factors and cytokines. The composition induces no immune rejection within 12 months of topical use. The dermal fibroblasts are human dermal fibroblasts. In some aspects, the composition further comprises human extra-cellular matrix proteins. In additional aspects, the composition further comprises glycerin. The keratinocytes may be cultured for 43 passages. The dermal fibroblasts may be cultured for 6 passages. The composition may be derived from human keratinocyte and fibroblast cell banks that contain no detectable pathogens. The cellularized scaffold construct may be rectangular in shape or trimmed to the shape of a wound bed. The cellularized scaffold construct may be for application to a single patient only. In some aspects, the composition may be prescribed for the treatment of adults with thermal burns in unit dosages, and the unit dosage is an approximately 100 cm² rectangle. The rectangular unit dosage may be approximately 8 cm by 12.5 cm. In some aspects, the composition may be prescribed for the treatment of adults with thermal deep partial-thickness burns in unit dosages, and the unit dosage is an approximately 100 cm² rectangle. The rectangular unit dosage may be approximately 8 cm by 12.5 cm. The composition provides durable wound closure at a thermal burn treatment site at 3 months without autograft placement. The composition may be a xenotransplantation product. In some aspects, mouse cells are not used in the manufacture of the composition. The composition may further include a packaging material gamma irradiated at a target minimum dose of 30.0 kGy. The composition has been tested using USP <87> (in vitro biological reactivity) and were found to exhibit no cytotoxicity. The composition has been tested using USP <88> (in vivo biological reactivity) and meet Class VI standards. The composition may be retained at 37+2° C. in a humidified, 5% CO₂ incubator for at least 25 days. In some aspects, the composition further includes a packaging layer having an oxygen transmission less than 0.003 cc/100 in²/24 hours. In some aspects, the composition further includes a packaging layer having a moisture permeation of less than 0.0065 g/100 cm²/24 hours. In some aspects, the composition further includes a 30 gauge layer aluminum foil pouch. In some aspects, the composition further includes an 18 pt cardboard carton comprising an opacity percentage of 99.74%. The composition may be stored inside a foil pouch until preparation for clinical use. The foil pouch does not contact the composition during packaging, shipping, or clinical use. The composition may be cryopreserved within the foil until preparation for clinical use. The composition may be substantially free of antimicrobial compositions selected from the group consisting of mafenide acetate, silver-containing antimicrobials, chlorhexidine solution, and combinations thereof. The composition may have improved efficacy treating adult thermal burns in the absence of antimicrobial compositions. In some aspects, a single subcutaneous injection of the keratinocytes into immunodeficient mice does not result in tumor formation by 23 weeks post-injection. In some aspects, a topical application of the composition on full-thickness excisional wounds in immunodeficient mice does not result in tumor formation by 20 weeks post-dose. In some aspects, no evidence of local or systemic toxicity is observed in immunocompromised athymic nude mice contacted with the composition.

Other aspects of the present disclosure include a method for preparing one or more cryopreserved constructs comprising allogeneic cultured keratinocytes for treatment of a patient in need thereof. The method may include: warming a hold solution in a warming device at 35° C. to 39° C.; pouring the warmed hold solution into a sterile hold dish; sterilely removing an insert tray containing the construct from a product dish; placing the insert tray into the hold dish; and maintaining the construct in the hold solution for 15 minutes to 4 hours.

The method may further include removing the construct from a polycarbonate membrane of the insert tray. The construct may include a dermal side and an epi-dermal side, wherein the construct is packaged with the dermal side in contact with the polycarbonate membrane. The method may further include meshing the construct up to a 1:1 ratio using a mesher. The mesher may be moistened as needed to prevent adhesion and maintain construct moisture using the hold solution, sterile 0.9% normal saline, or lactated Ringer's solution. The construct may be operable to be meshed with either the dermal side or the epidermal side up. The hold solution may be contained within a bottle stored in a laminated, foil pouch. The hold solution may be warmed in a warming oven/cabinet at 35° C. to 39° C. for at least 45 minutes prior to use. The hold solution may be warmed in a water bath at 35° C. to 39° C. for at least 15 minutes prior to use. The warmed hold solution may be poured into the sterile hold dish immediately after it is removed from the warming device. The hold dish may be initially packaged in a pouch. The product dish holding the insert tray containing the construct may be initially stored within a foil pouch and optionally further stored in a carton. The method may further include removing the carton from a freezer or dry ice. The method may further include opening the carton and foil pouch to re-move the product dish holding the insert tray containing the construct. The time between removing the carton from the freezer or dry ice and placing the insert tray in the hold dish may be no longer than 10 minutes. The insert tray may be removed from the product dish using either sterile forceps or sterile, gloved fingers. The insert tray may be placed in the hold dish beginning with one edge and lowering it to an opposite edge to minimize trapping bubbles beneath the insert tray. The method may further include preparing a wound bed via excision or debridement. The method may further include determining the number of constructs that need to be thawed. If two or more constructs are required, the two or more constructs are prepared in one or more batches. Preparation of one or more constructs takes approximately 20 minutes.

Aspects of the present disclosure may further include a method of treating a patient with deep partial-thickness burns comprising: preparing a wound bed via excision or debridement; placing one or more thawed and meshed constructs in contact with the prepared wound bed, the one or more constructs comprising allogeneic cultured keratinocytes and having a dermal side and an epidermal side the construct dermal side down; and anchoring the construct to the wound bed and/or surrounding tissue.

The method may further include applying a fibrin glue to the construct. Applying a fibrin glue to the construct does not result in cell death to the allogeneic cultured keratinocytes. Applying a fibrin glue to the construct may result in a less than 10% change in proliferation, migration, or secretion in the allogeneic cultured keratinocytes. Applying a fibrin glue to the construct may increase the percentage of skin graft take, wound bed adherence, or a combination thereof. Applying a fibrin glue to the construct may decrease side effects selected from the group consisting of bacterial infection, blood loss, postoperative pain, and combinations thereof. Applying a fibrin glue to the construct may result in a less than 10% decrease in the viability the allogeneic cultured keratinocytes. Applying a fibrin glue to the construct may result in a less than 30% decrease in the viability the allogeneic cultured keratinocytes. The method may further include abutting the two or more constructs when placing each construct on the wound bed. Two or more constructs may be used. If the wound bed to be treated is smaller than one construct, the method may further include trimming excess construct before or after anchoring the construct to the wound bed. The one or more constructs may have contact across the entire surface of the wound bed. The construct is not stretched or expanded during placement or anchoring so as to not degrade the structural integrity of the construct. The construct may be anchored with staples, tissue adhesive such as cyanoacrylate, or sutures. The method may further include placing a porous, nonadherent contact dressing over the one or more constructs and leaving the dressing in place for one week before changing. The method may further include placing a second layer of dressing that does not contain silver. The method may further include placing an outer bolster or wrap that keeps the one or more constructs from moving.

Aspects of the present disclosure may further include a method of treating an adult patient in need of surgical intervention comprising administering to an intact dermal element of the adult patient in need thereof an allogeneic cellularized scaffold, wherein the scaffold comprises dermal fibroblasts and murine collagen.

Hypersensitivity reactions may occur in less than 5% of the treated adult patients or in less than 1% of patients. The cellularized scaffold may be compatible with autograph meshing devices. The method may further include meshing the cellularized scaffold at ratios up to 1:1 prior to administering. The administering step may include topical administration to a prepared wound bed. The wound bed may be prepared by excision or debridement. In some aspects, more than one of cellularized scaffold constructs may be applied to cover the wound bed. The composition may be suitable for patients with no known allergies to murine collagen or products comprising ingredients of bovine or porcine origin. The cellularized scaffold construct may be trimmed to the shape of the wound bed. The cellularized scaffold construct may be for application to a single patient only. The method may further include contacting a healthcare provider if cellularized scaffold is dislodged. The method may further include disposing of unused cellularized scaffold as surgical biohazardous waste. The method may further include informing the adult patients that mouse cells were used in the early development of the cellularized scaffold. The method may further include informing the adult patients that cellularized scaffold comprises glycerin and could cause irritation if the patients are sensitive to glycerin.

Aspects of the present disclosure may further include a method of treating an adult patient for thermal burns comprising administering to an intact dermal element of the adult patient in need thereof an allogeneic cellularized scaffold, wherein the scaffold comprises dermal fibroblasts and murine collagen.

Aspects of the present disclosure may further include a method of treating an adult patient for deep partial-thickness burns comprising administering to an intact dermal element of the adult patient in need thereof an allogeneic cellularized scaffold, wherein the scaffold comprises dermal fibroblasts and murine collagen.

Aspects of the present disclosure may further include a method of treating adult patients in need of surgical intervention comprising applying a composition of allogeneic cultured keratinocytes comprising a cellularized scaffold construct to a surgically prepared wound bed, wherein the scaffold construct further comprises dermal fibroblasts and murine collagen.

Aspects of the present disclosure may further include a composition comprising a rectangular sheet of a viable, bioengineered, allogeneic cellularized scaffold product derived from keratinocytes grown on gelled collagen containing dermal fibroblasts.

Aspects of the present disclosure may further include a composition comprising two or more sheets of a viable, bioengineered, allogeneic cellularized scaffold product derived from keratinocytes grown on gelled collagen containing dermal fibroblasts, wherein the composition is suitable for the treatment of thermal burns without overlapping the edges of the sheets.

### BRIEF DESCRIPTION OF THE FIGURES

FIG. 1 depicts an outer carton and foil pouch packaging for a composition of the present disclosure.

FIG. 2 depicts a product dish containing a composition of the present disclosure.

FIG. 3 depicts a product dish and insert tray containing a composition of the present disclosure.

FIG. 4 depicts a bottle contain hold solution within a laminated foil pouch.

FIG. 5 depicts a hold dish contained within a clear pouch.

FIG. 6 depicts a hold dish handled in a sterile field.

FIG. 7 depicts a sterile operator (gray gloves) removing a sterile hold dish presented by the nonsterile operator (white gloves).

FIG. 8 depicts a sterile operator placing the hold dish in the sterile field (gray rectangle).

FIG. 9 depicts the removal of the foil pouch.

FIG. 10 depicts removing a closed product dish from the foil pouch.

FIG. 11 depicts the placement of the product dish in a nonsterile area.

FIG. 12 depicts a nonsterile operator aspetically pouring hold solution into the hold dish.

FIG. 13 depicts a nonsterile operator (white gloves) opening the product dish.

FIG. 14 depicts a sterile operator (gray gloves) removing the insert tray.

FIG. 15 depicts a sterile operator placing an insert tray into the hold dish.

FIG. 16 depicts meshing a composition of the present disclosure.

FIG. 17 depicts a graph showing the viability of tissue punches exposed to ARTISS fibrin glue. The yellow and red dashed lines, respectively, represent 10% and 30% reductions in viability from the control. Data are mean±stdev of n=5 models for each condition.

FIG. 18 depicts construct morphology of a composition of the present disclosure. Epidermal layer attached to a dermal equivalent containing fibroblast. The epidermis has a single layer of highly ordered basal keratinocytes below several layers of spinous cells. A distinct layer of granular cells with prominent intracellular keratohyalin granules is present between the stratum corneum and the spinous cells.

FIG. 19 shows the Stratum Corneum Lipid Lamellae from a composition of the present disclosure visualized by electron microscopy.

FIG. 20A shows a composition of the present disclosure at weeks 0-1.

FIG. 20B shows a composition of the present disclosure at weeks 2-3.

FIG. 20C shows a composition of the present disclosure at weeks 3-4.

### DETAILED DESCRIPTION

The present disclosure encompasses compositions comprising allogeneic cultured keratinocytes, methods of preparing such compositions for human application, and methods of administering such compositions. In particular, such compositions provide durable wound closure at a thermal burn treatment site, without autograft placement, when measured at 3 months. Such compositions also induce no immune rejection within 12 months of topical use.

### I. Compositions

One aspect of the present disclosure encompasses a composition of allogeneic cultured keratinocytes. Such compositions are designed for topical administration to a human, as detailed in Section III below. In a particular aspect, an allogeneic cultured keratinocyte composition of the present invention may be an allogeneic cellularized scaffold. In all embodiments disclosed herein, the composition is viable, and bioengineered (e.g. non-natural).

An allogeneic cultured keratinocyte composition of the present disclosure encompasses a fully stratified epithelial layer and a dermal equivalent layer (e.g. dermal layer). A composition of the present disclosure may be used as an organotypic human skin equivalent. Accordingly, the composition is a bioengineered (non-natural) bilayer tissue designed to mimic natural human skin with both an inner dermis-like layer and an outer epidermis-like layer. The epidermal layer is well-developed and comprises fully-stratified human keratinocytes that exhibit barrier function comparable to that of intact human skin.

The viable cells of a composition of the present disclosure (e.g., fibroblasts, NIKS cells, etc.) are metabolically active and secrete a spectrum of growth factors, chemotactic factors, cytokines, inflammatory mediators, enzymes, and host defense peptides that, after the composition is applied to a wound, may condition the wound bed, promote tissue regeneration and repair, and reduce infection. The viable cells of a composition of the present disclosure, namely keratinocytes and dermal fibroblasts, are karyotypically stable. In addition, the keratinocytes and dermal fibroblasts do not exhibit anchorage-independent growth.

In an exemplary embodiment, the composition is StrataGraft®™. In another exemplary embodiment, the composition is ExpressGraft™.

Generally speaking, a composition of the current disclosure may comprise any desired dimensions and surface area, limited only by the culture plates utilized. In particular embodiments, a composition of the present disclosure has a surface area of about 20 cm² to about 250 cm². In some embodiments, a composition of the present disclosure has a surface area of about 20 cm² to about 80 cm², about 80 cm² to about 140 cm², about 140 cm² to about 200 cm², or about 200 cm² to about 250 cm². In certain embodiments, a composition of the present disclosure has a surface area of about 90 cm² to about 110 cm². In one embodiment, a composition of the present disclosure has a surface area of about 100 cm².

A composition of the present disclosure may optionally be meshed. In some embodiments, a composition has a mesh ratio of about 1:1 or more (e.g., about 1.5:1, about 2:1, about 2.5:1, about 3:1, etc.). In some embodiments, a composition is not meshed.

Each of the layers of the composition is detailed below, along with other defining characteristics of the present compositions.

### Fully Stratified Epithelial Layer

A composition of the present disclosure comprises a fully stratified epithelial layer that is epidermis-like. The fully stratified epithelial layer comprises human keratinocytes. In some embodiments, the fully stratified epithelial layer comprises NIKS cells. NIKS® cells were deposited with the ATCC (CRL-12191) and are described in further detail in U.S. Pat. Nos. 5,989,837 and 6,964,869, the disclosures of which are incorporated herein by reference.

A fully stratified epithelial layer may encompass NIKS® cells engineered to express a variety of exogenous nucleic acids. Expressly contemplated are NIKS® cells engineered to express an exogenous gene encoding a VEGF protein (e.g., VEGF-A, etc.), an exogenous gene encoding a hypoxia-inducible factor (e.g., HIF-1A, etc.), an exogenous gene encoding an angiopoietin (e.g., ANGPT1, etc.), an exogenous gene encoding a cathelicidin peptide or a cleavage product thereof (e.g., hCAP-18, etc.), an exogenous gene encoding a beta-defensin (e.g., hBD-3, etc.), an exogenous gene encoding a keratinocyte growth factor (e.g., KGF-2, etc.), an exogenous gene encoding a tissue inhibitor of metalloproteinases (e.g., TIMP-1, etc.), an exogenous IL-12 gene, as well as exogenous nucleic acid sequences encoding other antimicrobials, growth factors, transcription factors, interleukins and extracellular matrix proteins. As non-limiting examples, see for instance, U.S. Pat. Nos. 7,498,167, 7,915,042, 7,807,148, 7,988,959, 8,808,685, 7,674,291, 8,092,531, 8,790,636, 9,526,748, 9,216,202, and 9,163,076, and US20190030130, the disclosures of which are incorporated herein by reference. Compositions comprising NIKS cells engineered to express an exogenous nucleic acid encoding a desired protein produce a greater amount of that protein (e.g., at least 10%, at least 20%, at least 30%, etc. more) than a composition comprising NIKS cells that do not contain the exogenous nucleic acid.

In some embodiments, the fully stratified epithelial layer has a thickness of about 75 μm to about 120 μm, as measured by histology. For example, the fully stratified epithelial layer may have a thickness of about 75, 80, 85, 90, 95, 100, 105, 110, 115, or 120 μm, as measured by histology.

### (b) a Dermal Equivalent Layer

A composition of the present disclosure encompasses a dermal equivalent layer that is dermis-like. The dermal equivalent layer has a top surface and a bottom surface, and comprises human dermal fibroblasts within a matrix.

### Dermal Fibroblasts

A composition of the present disclosure encompasses a dermal equivalent layer that comprises dermal fibroblasts. In most embodiments, the dermal fibroblasts are human fibroblasts. In exemplary embodiments, the human dermal fibroblasts are primary normal human dermal fibroblasts.

### Matrix

A composition of the present disclosure encompasses a dermal equivalent layer that comprises a matrix. The matrix of the dermal equivalent layer comprises human collagen and optionally, murine type I collagen. In some embodiments, the matrix of the dermal equivalent layer comprises human extracellular matrix proteins, such as human type I collagen, human type III collagen, human type IV collagen, human type VI collagen, and optionally, murine type I collagen.

The collagen present in the dermal equivalent may include type I murine collagen. Alternatively, the only collagen present in the dermal equivalent may be produced by cells of the skin substitute (e.g., human dermal fibroblasts). The matrix may further comprise additional biomolecules produced by the cells contained therein. In an exemplary embodiment, the dermal layer is composed of normal human dermal fibroblasts embedded within a matrix produced and organized by the fibroblasts (e.g. an extracellular matrix). In some iterations of this embodiment, there is no non-human collagen in the dermal equivalent layer. In other iterations of this embodiment, there is up to about 85% non-human collagen in the dermal equivalent layer. In particular iterations, the non-human collagen is murine. In another iteration, the non-human collagen consists of murine collagen and includes no other non-human collagen material. In another exemplary embodiment, the dermal equivalent layer is composed of normal human dermal fibroblasts embedded in a gelled-collagen matrix that contains purified murine type I collagen. For the avoidance of doubt, in this embodiment, although the murine type I collagen is gelled to give the dermal layer its primary structure, the normal human dermal fibroblasts embedded therein may produce and contribute collagen (and other biomolecules) to the matrix. Accordingly, the collagen matrix may comprise both murine type 1 collagen and human collagen (produced from the human dermal fibroblasts).

A composition of the present disclosure may comprise human type I collagen and murine type I collagen, wherein the murine type I collagen is not more than 90% by weight of total collagen in the composition. For instance, in some embodiments, the murine type 1 collagen is about 60% to about 90% by weight of the total type collagen in the composition.

### Biological Reactivity

Generally speaking, a composition of the present disclosure comprises no detectable pathogens. In some embodiments, a composition of the present disclosure is derived from human keratinocyte and fibroblast cell banks that contain no detectable pathogens.

In particular embodiments, a composition of the present disclosure has been tested using USP <87> (in vitro biological reactivity) and was found to exhibit no cytotoxicity. In another particular embodiment, a composition of the present disclosure has been tested using USP <88> (in vivo biological reactivity) and met Class VI standards. In preferred embodiments, a composition of the present disclosure has been tested using USP <87> (in vitro biological reactivity) and was found to exhibit no cytotoxicity and has been tested using USP <88> (in vivo biological reactivity) and met Class VI standards. The protocol for USP <87> and USP <88> are known in the art.

A composition of the present disclosure has improved efficacy for treating adult thermal burns in the absence of antimicrobial compositions. That is, a composition of the present disclosure is substantially free of antimicrobial compositions and does not require antimicrobial compositions for efficacy in treating adult thermal burns. Examples of such antimicrobial compositions include mafenide acetate, silver-containing antimicrobials, chlorhexidine solution, and combinations thereof.

### Allogeneic Cellularized Scaffold

An allogeneic cultured keratinocyte composition of the present invention may be an allogeneic cellularized scaffold. As used herein, an “allogeneic cellularized scaffold” is a sterile composition of the present disclosure that has been cryopreserved, prepared for application to a patient, and optionally, dimensioned for placement over a wound bed of the patient. The phrase “allogeneic cellularized scaffold” may be used interchangeably herein with the term “construct.”

An allogeneic cellularized scaffold comprises dermal fibroblasts and murine collagen as detailed above.

### Manufacturing

Suitable manufacturing processes for producing a composition of the present disclosure have been previously described in the art. See, for instance, U.S. Pat. Nos. 7,498,167, 7,915,042, 7,807,148, 7,988,959, 8,808,685, 7,674,291, 8,092,531, 8,790,636, 9,526,748, 9,216,202, 9,163,076, 10,091,983, and US20190030130, the disclosures of which are each incorporated by reference in their entirety.

In each of the above embodiments, mouse cells are not used in the manufacture of a composition of the present disclosure. Regardless, a composition of the present disclosure may be considered a xenotransplantation product under certain regulatory definitions due to how cells of the composition were historically cultured.

### Cryopreservation

Each of the compositions disclosed herein may be cryopreserved. Methods of suitable cryopreservation are disclosed, for instance, in U.S. Pat. No. 10,091,983, herein incorporated by reference in its entirety. Cryopreserved compositions may comprise glycerin.

After a composition of the present disclosure is thawed following cryopreservation, the composition may secrete a plurality of proteins including human growth factors and cytokines. For instance, a thawed composition may secrete a protein selected from bFGF, GM-CSF, HGF, IL-1α, IL-6, IL-8, IL-10, MMP-1, MMP-3, MMP-9, PIGF, SDF-1α, TGF-β1, and VEGF-A.

### Packaging

Each of the compositions disclosed here may be packaged, for instance, for delivery from the site of manufacture to the site of the end-user. Packaging material used herein may be gamma irradiated at a target minimum dose of 30.0 kGy.

In some embodiments, a composition may be packaged in a packaging layer having an oxygen transmission rate of less than 0.003 cc/100 in²/24 hours.

In some embodiments, a composition may be packaged in a packaging layer having a moisture permeation rate of less than 0.0065 g/100 cm²/24 hours.

In particular embodiments, a composition may be packaged in a packaging layer having an oxygen transmission less than 0.003 cc/100 in²/24 hours and a moisture permeation rate of less than 0.0065 g/100 cm²/24 hours.

In certain embodiments, a composition of the present disclosure may be packaged and stored inside a foil pouch until preparation for clinical use. Such a foil pouch may comprise a 30 gauge layer of aluminum foil. Generally speaking, the foil pouch does not contact the composition during packaging, shipping, or clinical use. In particular embodiments, the composition is cryopreserved within the foil pouch until preparation for clinical use.

In some embodiments, a foil pouch comprising a composition of the present disclosure may be stored in an 18 pt cardboard carton comprising an opacity percentage of 99.74%. For instance, a composition of the present disclosure may be cryopreserved within a foil pouch and stored in an 18 pt cardboard carton comprising an opacity percentage of 99.74% until preparation for clinical use.

### Dosing

Generally speaking, a unit dosage of the compositions described herein is approximately a 100 cm² rectangle. However, those of skill in the art will recognize that the unit dosage may be altered by altering the size and/or shape of the insert tray during manufacture. In some embodiments, the rectangular unit dosage is approximately 8 cm by 12.5 cm. In all embodiments, a unit dosage may be trimmed to the shape of a wound bed.

A patient may be prescribed one or more unit dosages. In particular embodiments the unit dosage for treatment of a thermal burn may be one or more 100 cm² rectangles. For instance, the unit dosage for treatment of a thermal deep partial-thickness burn may be one or more 100 cm² rectangles.

In particular embodiments, a unit dosage is for application to a single patient only.

### II. Methods of Preparing

Another aspect of the present disclosure encompasses methods of preparing a composition disclosed herein for application to a patient. In all embodiments disclosed in this section, a composition of allogeneic cultured keratinocytes as detailed in Section I above is cryopreserved and packaged for shipment to an end-user. Methods of suitable cryopreservation are disclosed, for instance, in U.S. Pat. No. 10,091,983, herein incorporated by reference in its entirety.

Suitable packaging is described in Section I above. Generally speaking, a composition of the present disclosure is packaged in an insert tray held within a product dish. The cells of the composition only come into contact with the insert tray, but the product dish may comprise liquid that contacts the cells held within the insert tray. In some embodiments, the insert tray comprises a polycarbonate membrane that supports the allogeneic cultured keratinocyte composition. In certain embodiments, an allogeneic cultured keratinocyte composition comprises a dermal side and an epidermal side. In these embodiments, the composition is placed within the insert tray such that the dermal side is in contact with the polycarbonate membrane. In certain embodiments, the hold solution is packaged in a bottle or equivalent container and sealed in a pouch, such as a foil pouch.

In some embodiments, the present disclosure encompasses methods for preparing one or more cryopreserved compositions comprising allogeneic cultured keratinocytes for application to a patient in need of treatment for a thermal burn. The method generally comprises warming a hold solution in a warming device, pouring the warmed hold solution into a sterile hold dish, sterilely removing an insert tray containing the allogeneic cultured keratinocyte composition from a product dish, placing the insert tray into the hold dish, and maintaining the composition in the hold solution for up to 4 hours.

The warming device used to warm the hold solution should maintain a temperature between about 32° C. and 41° C. Suitable non-limiting examples of warming devices may include a water bath, a warming oven, or a warming cabinet. For instance, a warming device may maintain a temperature of about 32, 33, 34, 35, 36, 37, 38, 39, 40, or 41° C. In some embodiments, a warming device maintains a temperature between about 32° C. to about 41° C., about 33° C. to about 40° C., about 34° C. to about 40° C., or about 35° C. to about 39° C. In a particular embodiment, a warming device may maintain a temperature of between about 35° C. to 39° C.

In some embodiments, the composition is maintained in the hold solution for about 15, 30, 45, 60, 75, 90, 105, 120, 135, 150, 165, 180, 195, 210, 225, or 240 minutes. In other embodiments, the composition is maintained in the hold solution for up to 1 hour up to 2 hours, up to 3 hours, or up to 4 hours. In still other embodiments, the composition is maintained in the hold solution for between about 15 minutes and 4 hours.

In some embodiments, a method of the present disclosure comprises warming a hold solution in a warming device at 35° C. to 39° C.: pouring the warmed hold solution into a sterile hold dish; sterilely removing an insert tray containing the construct from a product dish; placing the insert tray into the hold dish; and maintaining the construct in the hold solution for 15 minutes to 4 hours.

In some embodiments, a method of present disclosure further comprises removing the allogeneic cultured keratinocytes from a polycarbonate membrane of the insert tray. In such embodiments, a method of the present disclosure comprises warming a hold solution in a warming device at 35° C. to 39° C.; pouring the warmed hold solution into a sterile hold dish; sterilely removing an insert tray containing the construct from a product dish; placing the insert tray into the hold dish; maintaining the construct in the hold solution for 15 minutes to 4 hours, and removing the allogeneic cultured keratinocytes from a polycarbonate membrane of the insert tray. Such removal may be done with gloved fingers, or a pair of atraumatic forceps, for example.

In particular embodiments, an allogeneic cultured keratinocyte composition of the present disclosure may be meshed before being applied to a patient. For instance, the composition may be meshed up to a 1:1 ratio using a mesher. In some embodiments, the mesher is moistened as needed to prevent adhesion and to maintain the moisture of the composition using a hold solution, sterile 0.9% normal saline, or lactated Ringer's solution. Generally speaking, if a composition comprises a dermal side and an epidermal side, the composition may be meshed with either the dermal side or the epidermal side up.

In certain embodiments, a method of the present disclosure comprises warming a hold solution in a warming device at 35° C. to 39° C.; immediately pouring the warmed hold solution into a sterile hold dish; sterilely removing an insert tray containing the construct from a product dish; placing the insert tray into the hold dish: maintaining the construct in the hold solution for 15 minutes to 4 hours, and removing the allogeneic cultured keratinocytes from a polycarbonate membrane of the insert tray.

In some embodiments, a method of the present disclosure comprises first removing the product dish holding the insert tray from a freezer or dry ice. In some instances, the product dish is packaged in a foil pouch, as detailed above. The foil pouch may be stored in the freezer or dry ice in a carton, as detailed above. Such embodiments further comprise opening the carton and removing the foil pouch encompassing the product dish. Generally speaking, the time between removing the carton from the freezer or dry ice and placing the insert tray in the hold dish should not be longer than 15 min. In some embodiments, the time is not longer than 5, 6, 7, 8, 9, 10, 11, 12, 13, 14, or 15 minutes. In preferred embodiments, the time is not longer than 10 minutes.

An insert tray may be sterilely removed from the product dish using sterile forceps, sterile gloved fingers, or similar sterile means. Regardless of the sterile method used, the insert tray is preferentially placed in the hold dish beginning with one edge and lowering it to an opposite edge to minimize trapping bubbles beneath the insert tray. If bubbles are present, the insert tray should be gently lifted from the hold dish and slowly lowered back down into the hold dish.

A method of the present disclosure for preparing one or more cryopreserved compositions comprising allogeneic cultured keratinocytes for application to a patient in need of treatment for a thermal burn may further comprise determining the number of compositions that need to be prepared. If more than one composition needs to be prepared, the compositions may be prepared in one batch, or more than one batch. In some embodiments, the preparation of one or more compositions may take between about 18 minutes and 4 hours. In preferred embodiments, the preparation of one or more compositions may take approximately 20 minutes.

Before application of a composition comprising allogeneic cultured keratinocytes to a patient, an appropriate wound bed should be prepared on the patient. For instance, a wound bed may be prepared via excision or debridement.

### III. Methods of Administering

Yet another aspect of the present disclosure encompasses methods of administering a composition described in section I above to treat thermal burns. In one embodiment, the present disclosure encompasses methods of treating a patient with deep partial-thickness burns. For instance, the present disclosure encompasses methods of treating a patient with thermal burns containing intact dermal elements for which surgical intervention is clinically indicated. Such methods comprise preparing a wound bed via excision or debridement; placing one or more prepared and optionally meshed allogeneic cultured keratinocyte compositions in contact with the prepared wound bed, and anchoring the one or more compositions to the wound bed and/or surrounding tissue.

Suitable methods of anchoring an allogeneic cultured keratinocyte composition may include staples, tissue adhesive such as cyanoacrylate, sutures, and fibrin glue. In some embodiments, the fibrin glue is applied to the composition before the composition is placed on the wound bed. In these embodiments, applying a fibrin glue to the composition does not result in cell death to the allogeneic cultured keratinocytes. In some embodiments, applying a fibrin glue to the composition results in a less than 10% change in proliferation, migration, or secretion in the allogeneic cultured keratinocytes. In a particular embodiment, applying a fibrin glue to the composition increases the percentage of skin graft take, wound bed adherence, or a combination thereof. In another particular embodiment, applying a fibrin glue to the composition decreases one or more side effects selected from the group consisting of bacterial infection, blood loss, postoperative pain, and combinations thereof. In still another preferred embodiment, applying a fibrin glue to the composition results in a less than 30%, less than 20% or less than 10% decrease in the viability the allogeneic cultured keratinocytes. For instance, applying a fibrin glue to the composition may result in less than 30, 29, 28, 27, 26, 25, 24, 23, 22, 21, 19, 18, 17, 16, 15, 14, 13, 12, 11, 10, 9, 8, 7, 6, 5, 4, 3, 2, or 1% decrease in the viability of the allogeneic cultured keratinocytes. In particular embodiments, applying a fibrin glue to the composition may increase the viability of the allogeneic cultured keratinocytes.

One or more compositions may be used in a method of the present disclosure. For instance, 1, 2, 3, 4, 5, 6, 7, 8, 9, 10, or more than 10 compositions may be used to cover one or more wound beds on a subject. If more than one composition is used, the compositions may be abutted to each other. While two or more compositions may be overlapped, it is not necessary. Generally speaking, if more than one composition is used, the compositions are abutted to each other such that they have contact across the entire surface of the wound bed. If a particular wound bed is smaller than a single composition, the composition may be trimmed before or after anchoring to remove excess composition. As used herein, “excess” refers to composition that is not needed to cover a wound bed or anchor the composition to the wound bed.

Typically, a composition is placed and anchored so as to not degrade the structural integrity of the construct. In this regard, care should be taken to avoid stretching or expanding the composition in such a way that the structural integrity of the composition is degraded.

After a composition of the present invention is placed and anchored, it may be covered by one or more dressings, bolsters, or wraps. For instance, a method of the present disclosure may further comprising placing a porous, nonadherent contact dressing over the one or more compositions. In certain embodiments, a method of the present disclosure may further comprise playing a second layer of dressing over a first dressing. Generally speaking, such a dressing should not include silver. Dressings may be left in place for 1, 2, 3, 4, 5, 6, 7, 8, 9, or 10 days. For instance, dressings may be left in place for between about 4 and about 8 days. In some instances, dressings may be left in place for about one week.

A method of the present disclosure may also comprise placing an outer bolster or wrap over the one or more dressings that keeps the one or more compositions from moving. A method of the present disclosure may further comprise contacting a healthcare provider if a placed composition is dislodged.

In further embodiments, a method of the present disclosure may comprise disposing of unused composition as surgical biohazardous waste.

In some embodiments, a composition may be meshed at ratios up to 1:1 prior to placement.

In most embodiments, hypersensitivity reactions after placement of a composition described herein occur in less than 10% of the treated adult patients. In some embodiments, hypersensitivity reactions occur in less than 5% of the treated adult patients. In other embodiments, hypersensitivity reactions occur in less than 1% of patients.

Compositions disclosed herein may be suitable for patients with no known allergies to murine collagen or products comprising ingredients of bovine or porcine origin. In certain embodiments, a method of the present disclosure encompasses informing the adult patients that mouse cells were used in the early development of the cellularized scaffold. In other embodiments, methods of the present disclosure may comprise informing the adult patients that cellularized scaffold comprises glycerin and could cause irritation if the patients are sensitive to glycerin.

Such information may be relayed to the patient or the patient's representative before placement of the composition.

In each of the above embodiments, the composition may be an allogeneic cellularized scaffold as detailed in section I above.

In specific embodiments, the present disclosure encompasses a method of treating an adult patient in need of surgical intervention for treatment of a wound comprising an intact dermal element, the method comprising administering to the wound an allogeneic cellularized scaffold, wherein the scaffold comprises dermal fibroblasts and murine collagen.

In other specific embodiments, the present disclosure encompasses a method of treating an adult patient for thermal burns comprising administering to an intact dermal element of the adult patient in need thereof an allogeneic cellularized scaffold, wherein the scaffold comprises dermal fibroblasts and murine collagen.

In still other specific embodiments, the present disclosure encompasses a method of treating an adult patient for deep partial-thickness burns comprising administering to an intact dermal element of the adult patient in need thereof an allogeneic cellularized scaffold, wherein the scaffold comprises dermal fibroblasts and murine collagen.

In yet other specific embodiments, the present disclosure encompasses a method of treating adult patients in need of surgical intervention comprising applying a composition of allogeneic cultured keratinocytes comprising a cellularized scaffold construct to a surgically prepared wound bed, wherein the scaffold construct further comprises dermal fibroblasts and murine collagen.

### Definitions

As used herein, a “hold solution” refers to a cell media comprising a source of nutrients and osmotic regulators. A hold solution is used to thaw a cryopreserved composition of the present disclosure and/or remove cryoprotectant from the composition. In some embodiments, the hold solution comprises 4-(2-hydroxyethyl)-1-piperazineethanesulfonic acid. In particular embodiments, the hold solution comprises an F-12 nutrient mixture. Suitable hold solutions are further described in US application xxx, herein incorporated by reference.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL” The endpoint may also be based on the variability allowed by an appropriate regulatory body, such as the FDA, USP, etc.

As used herein, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including.” it is understood that direct support should be afforded also to “consisting essentially of” language as well as “consisting of” language as if stated explicitly and vice versa.

As used herein, the term “sterile” refers to a skin equivalent that is essentially or completely free of detectable microbial or fungal contamination.

As used herein, the term “NIKS cells” refers to cells having the characteristics of the cells deposited as cell line ATCC CRL-12191. “NIKS” stands for near-diploid immortalized keratinocytes and is a registered trademark.

As used herein, the term “viable” when used in reference to a skin equivalent refers to the viability of cells in the skin equivalent following cryopreservation. In preferred embodiments, a “viable” skin has an A550 of at least 50%, 60%, 70%, 80% or 90% of a control non-cryopreserved tissue as measured by an MTT assay or at least 50%, 60%, 70%, 80% or 90% of the readout value of a similar viability assay.

As used herein, the terms “tissue container,” “tissue container assembly,” “product dish,” and “hold dish” are used interchangeably to refer to a container used to store, transport, and/or thaw a tissue (e.g. an allogeneic cellularized scaffold product). Multiple tissue containers may be used in the processes of storing and thawing the tissue. For example, an allogeneic cellularized scaffold product may be cryopreserved and stored in a first tissue container and a second tissue container may be configured to connect to the first tissue container to act as a lid to the first tissue container, such that a tissue container assembly includes the first and second containers acting as identical top and bottom portions. In addition, a hold dish may be a tissue container without an allogeneic cellularized scaffold product stored within it. The hold dish may include two tissue containers acting as identical top and bottom portions.

As used herein, the terms “tray” and “insert tray” are used interchangeably to refer to a tray or dish operable to hold the allogeneic cellularized scaffold product and be inserted into, fit within, or rest inside a tissue container.

### EXAMPLES

The following examples illustrate various iterations of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### Example 1: Description

STRATAGRAFT is a viable, bioengineered, allogeneic, cellularized scaffold product that contains a fully-stratified epithelial layer comprised of differentiated, multilayered, epidermal keratinocytes from a single human donor. The keratinocytes are grown on a purified murine Type I collagen matrix embedded with fibroblasts from a second human donor (i.e. separately sourced). STRATAGRAFT is produced from well-characterized human keratinocyte and fibroblast cell banks that contain no detectable pathogens. The cells are metabolically active allogeneic NIKS keratinocytes and dermal fibroblasts. In one embodiment, the STRATAGRAFT may comprise NIKS keratinocytes and human dermal fibroblasts that are cultured for 43 and 6 passages, respectively, and do not exhibit anchorage-independent growth (a standard assay that evaluates the potential for cellular transformation). In another embodiment, STRATAGRAFT may comprise NIKS keratinocytes and human dermal fibroblasts that are at passage 40 and 7, respectively, and do not exhibit anchorage-independent growth. The NIKS keratinocytes and human dermal fibroblasts contained in STRATAGRAFT may be karyotypically stable. In another embodiment, The epidermal layer of STRATAGRAFT is generated from NIKS keratinocytes, which originate from normal keratinocytes (BC-1-Ep) isolated from neonatal human foreskin.

STRATAGRAFT is produced from well-characterized human keratinocyte and fibroblast cell banks that contain no detectable pathogens. The cellular components of STRATAGRAFT have been extensively tested for tumorigenicity In one embodiment, the tumorigenic potential of STRATAGRAFT is evaluated by the karyotypic stability of the NIKS keratinocytes and human dermal fibroblasts. In another embodiment, the tumorigenic potential of STRATAGRAFT is evaluated by a single subcutaneous injection of NIKS keratinocytes into immunodeficient mice, which does not result in tumor formation by 23 weeks post-injection (i.e., a positive indication of safety). In further embodiment, the tumorigenic potential of STRATAGRAFT is evaluated by topical application of STRATAGRAFT on full-thickness excisional wounds in immunodeficient mice, which does not result in tumor formation by 20 weeks post-dose (i.e., a positive indication of safety). In another embodiment, the toxicity or tumorigenic potential of STRATAGRAFT is evaluated by immunocompromised athymic nude mice exposed to STRATAGRAFT. More specifically, in one embodiment, toxicity of STRATAGRAFT is evaluated by administration/application of 14 cm² to athymic nude mice (approximately 25% Total Body Surface Area) of STRATAGRAFT to full-thickness excisional wounds, followed by assessment for local or systemic toxicity from 90 to 140 days (13 to 20 weeks), wherein no evidence of local or systemic toxicity is observed (i.e., a positive indication of safety).

STRATAGRAFT is considered to be a xenotransplantation product because the keratinocyte component of STRATAGRAFT was originally derived in the presence of a mouse cell line. However, mouse cells are no longer used to manufacture STRATAGRAFT. No mouse cells or mouse-derived infectious agents are detectable in STRATAGRAFT.

STRATAGRAFT product manufacture includes reagents derived from animal materials including murine collagen, calf serum, porcine trypsin and purified bovine serum albumin.

STRATAGRAFT construct is loosely adherent to a supportive polycarbonate membrane insert and treated in glycerin-containing media. Each cryopreserved STRATAGRAFT construct is supplied with Hold Solution and Hold Dish, which are used for preparing STRATAGRAFT. The Hold Solution is a cell-culture medium that is not supplemented with growth factors.

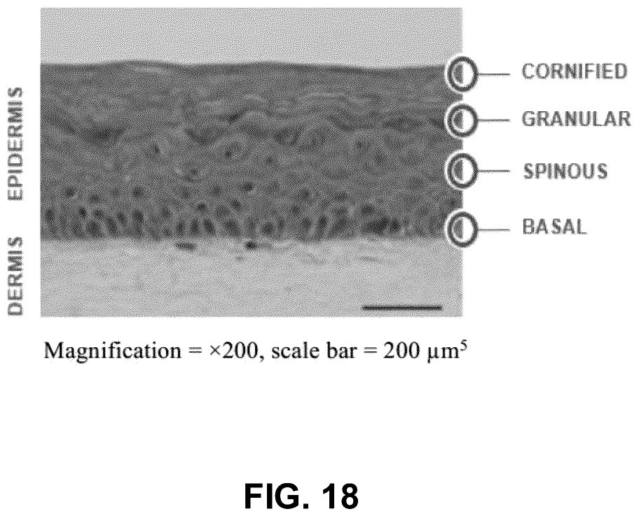

STRATAGRAFT has several structural and biological properties of interfollicular epidermis of human skin. The stratified epidermal layer in STRATAGRAFT that closely resembles interfollicular epidermis of human skin is produced by in vitro organotypic culture of NIKS keratinocytes. Immunohistochemistry and indirect immunofluorescence analysis of STRATAGRAFT revealed typical epidermal morphology containing a single basal layer of cuboidal cells with a high nucleus-to-cytoplasm ratio. The basal layer is present below stratified spinous, granular, and cornified layers. After hematoxylin and eosin staining, the granular layer showed distinctly stained keratohyalin granules, and a well-defined enucleated stratum corneum, characteristic of a mature epidermis. FIG. 18 depicts the morphology of STRATAGRAFT construct fixed, embedded, and stained with hematoxylin and eosin.

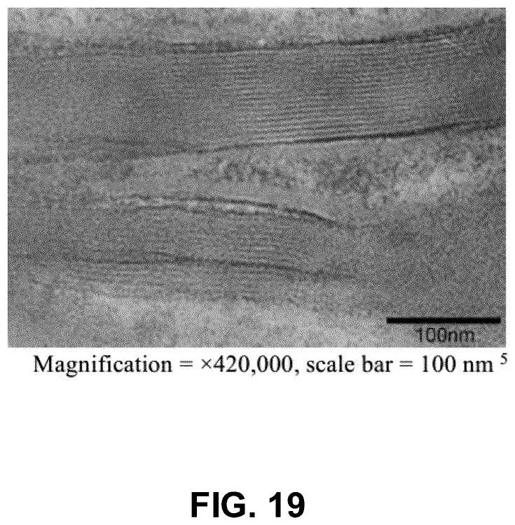

The formation of a competent epidermal permeability barrier within the stratum corneum depends on the assembly of specialized lipid lamellae that fill the intercorneocyte extracellular space. When stained with ruthenium tetroxide, STRATAGRAFT exhibits the alternating electron dense and lucent staining pattern that is characteristic of lipid lamellae (FIG. 19).

The integrity of STRATAGRAFT construct's barrier function was assessed by measuring skin SEI. The results demonstrated that STRATAGRAFT exhibited barrier function comparable to that of native human skin.

### Manufacturing

STRATAGRAFT was manufactured through organotypic culture under aseptic conditions in compliance with Good Manufacturing Practices. The process for producing STRATAGRAFT was optimized to promote epidermal stratification and barrier function. Prior to clinical use, STRATAGRAFT lots were subjected to proprietary testing to ensure quality, sterility, identity, purity, and potency.

Cryopreservation of STRATAGRAFT maintains cell viability and metabolic activity during the 12-month shelf life. STRATAGRAFT must be maintained in ultracold conditions (e.g., in a freezer at −70° C. to −90° C. (−94° F. to −130° F.) or on dry ice) during transport, storage, and transfer to the operating room until initiation of the thaw process. For example, it is shipped on dry ice and stored between −70° C. and −90° C. The Hold Solution and Hold Dish, are packaged separately and shipped at ambient temperature.

### Application

STRATAGRAFT is a viable, bioengineered, allogeneic, cellularized scaffold product. Each construct is for application to a single patient only. STRATAGRAFT is compatible with autograft meshing devices (crushing or noncrushing) and can be meshed at ratios up to 1:1. STRATAGRAFT should not be stretched or expanded because this may degrade its structural integrity.

STRATAGRAFT should have contact across the entire surface of the wound bed and can be anchored using adhesives, staples, or sutures to keep STRATAGRAFT from dislodging. If multiple constructs are required to cover large wound area, abutting the constructs is advised rather than overlapping.

### Mechanism of Action

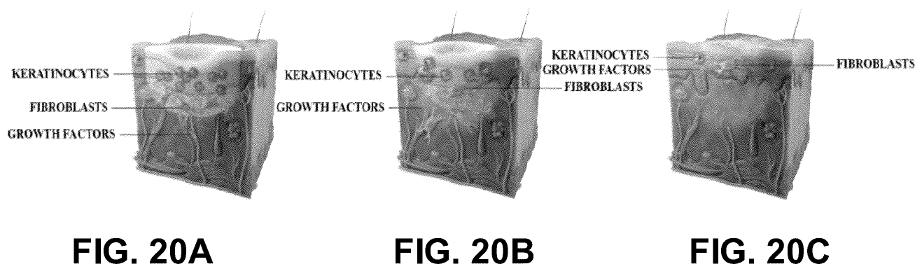

STRATAGRAFT is an allogeneic cellularized scaffold product containing metabolically active cells that produce and secrete a variety of growth factors, cytokines and human extracellular matrix proteins, which are known to be involved in wound repair and regeneration (FIGS. 20A-20C). STRATAGRAFT does not engraft and is replaced by the patient's own cells over time, eliminating or reducing the need for autografting to attain definitive closure of the majority of treated wounds.

The uppermost layers of the epidermal component serve as a physical barrier to microorganisms and water loss. STRATAGRAFT provides viable cells that interact with the local wound environment to produce and secrete a variety of growth factors, cytokines, and peptides. The NIKS keratinocytes in the epidermal layer produce normal adhesion proteins (e.g., integrins and cadherins) that enable tight adherence between the cells and also to the dermal equivalent. These adhesions confer handling characteristics to STRATAGRAFT, which make it amenable to meshing and secured placement, as is done regularly with splitthickness skin grafts. However, as discussed earlier, STRATAGRAFT should not be stretched or expanded because this may disrupt the structural integrity of the construct.

STRATAGRAFT does not heal by engraftment. During the phase 1b (STRATA2011) and phase 3 (STRATA2016) studies, biopsies taken 3 months after placement did not contain detectable DNA from the allogeneic cells of STRATAGRAFT. Alleles detected in DNA from STRATAGRAFT-treated sites matched previous patient samples, indicating healing by the subject's own cells and turnover of STRATAGRAFT throughout wound-healing. No STRATAGRAFT DNA remained at the treatment site in any subjects 3 months after STRATAGRAFT placement.

### Bioactive Factors Produced

Many bioactive factors (growth factors, cytokines, chemokines, and proteinases) produced by STRATAGRAFT are associated with various stages of wound-healing.

After thawing STRATAGRAFT following clinical instructions for use, in vitro testing identified secretion of growth factors, cytokines, and proteinases (bFGF, HGF, PIGF, TGF-β1, VEGF-A, GM-CSF, IL-1α, IL-6, IL8, IL-10, MMP-1, MMP-3, MMP-9, and SDF-1α [CXCL12]) by viable cells of STRATAGRAFT into surrounding media.

Following meshing, changes in the magnitude of several growth factors, enzymes, and cytokines were observed at 1 hour, and up to 7 days, after in vitro reculture.

### Bioresponsive Nature

Changes to the magnitude of growth factor, cytokine, and proteinase secretion were observed in response to physical perturbation of STRATAGRAFT in vitro. After thawing, STRATAGRAFT was either meshed at a 1:1 ratio or remained intact prior to a 7-day in vitro experiment. Bioactive factor secretion was measured starting at 1 hour, and up to 7 days after meshing. STRATAGRAFT secreted different levels of bioactive factors in response to the external mechanical stimulation of meshing. Changes in secretion of factors such as bFGF, IL-1α, SDF-1α, and MMP-9 were observed within 1 hour of meshing, indicating cells within STRATAGRAFT are bioactive in response to external cues.

### Immune Profile

STRATAGRAFT has been tested for immunological response. In a phase 1/2 study, STRATAGRAFT did not induce additional acute inflammatory cell infiltrates compared to the standard of care control, allograft. In the same study, PBMCs collected from patients did not exhibit increased responsiveness against NIKS keratinocytes after exposure to STRATAGRAFT. Patient PBMCs also exhibited similar natural killer cell-mediated killing of NIKS keratinocytes in vitro before and after exposure to STRATAGRAFT. Testing against a healthy, unburned, subject population confirmed these results were not misrepresented due to burn patient's immunocompromised status.

In a phase 3 pivotal trial (STRATA2016), prior to treatment, 4.3% of patients had reactivity to alleles found in STRATAGRAFT and 5.7% of patients had reactivity to alleles not found in STRATAGRAFT. At Month 3, 24.2% of patients had reactivity to alleles found in STRATAGRAFT and 30.6% of patients had reactivity to alleles not found in STRATAGRAFT. Although there is no known clinical significance to the development of antibodies to BSA, anti-BSA antibody levels increased in 22.4% of STRATAGRAFT-treated patients from baseline to Month 3.8 Overall, the observed reactivities were not targeted to the specific alleles of the cells in STRATAGRAFT. These observations were consistent with broad sensitization and/or heterologous sensitization seen in burn patients as they often receive other allogeneic products for standard treatment of severe burns.

### Absence of Langerhans Cells

STRATAGRAFT does not contain dendritic cells such as Langerhans cells. Langerhans cells are often considered the only cell type within the healthy epidermis capable of expressing major histocompatibility complex class II antigen. Engineered skin tissues that do not contain Langerhans cells or leukocytes (professional antigen presenting cells) greatly reduce the possibility of allograft antigen presentation.

### Example 2: Usage

STRATAGRAFT is an allogeneic cellularized scaffold product indicated for the treatment of adults with thermal burns containing intact dermal elements for which surgical intervention is clinically indicated (deep partial-thickness burns).

STRATAGRAFT is for topical application to a prepared wound bed (excision/debridement). A STRATAGRAFT construct is an approximately 100 cm² (approximately 8 cm by 12.5 cm) off-white rectangle. A STRATAGRAFT construct may be trimmed to fit the shape and size of the wound area. The surface area of STRATAGRAFT to be applied should be equal to the surface area of the wound to be treated. Multiple constructs may be applied to cover large wound areas. If multiple constructs are required to cover the wound area, abut or adjoin the STRATAGRAFT constructs, and it is not necessary to overlap the edges. Each construct is for application to a single patient only.

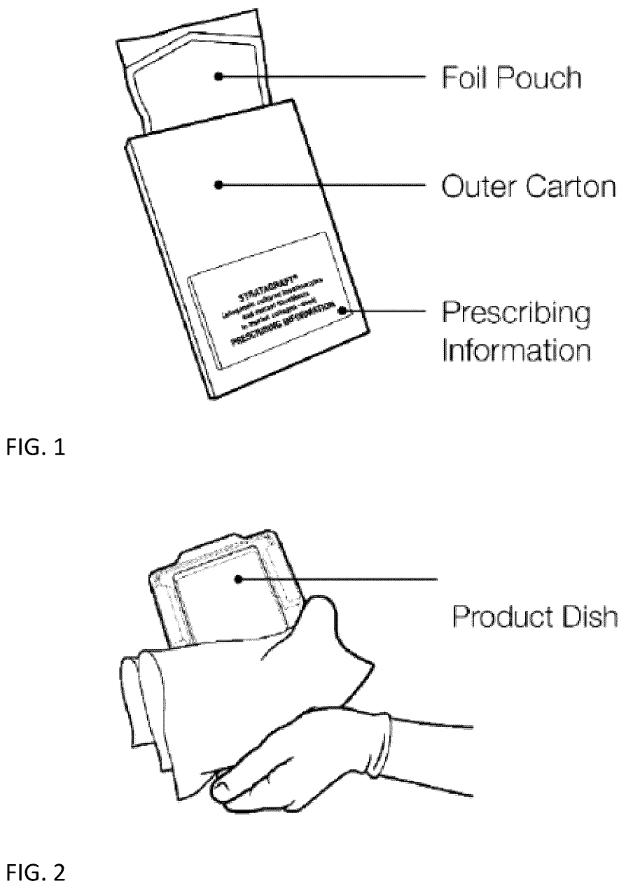

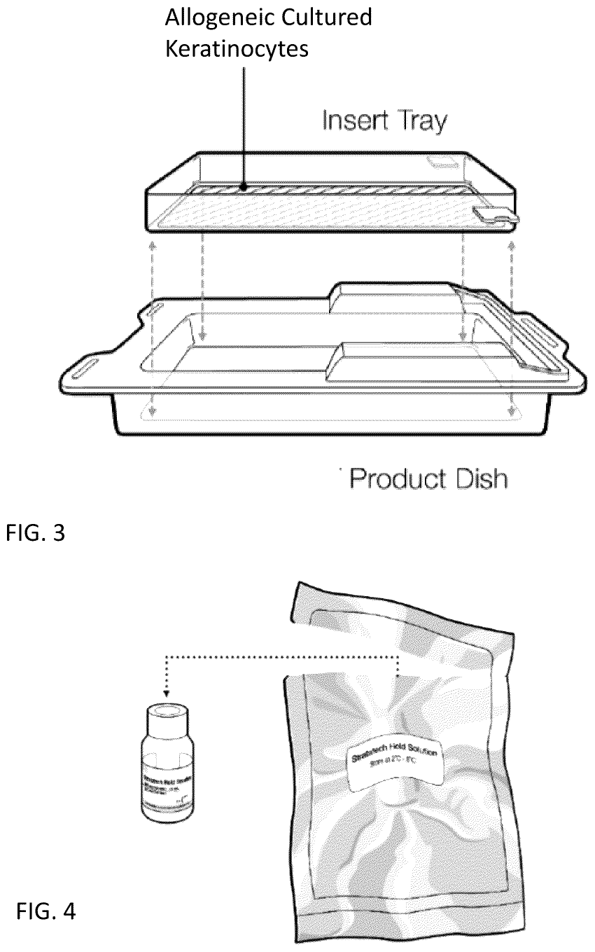

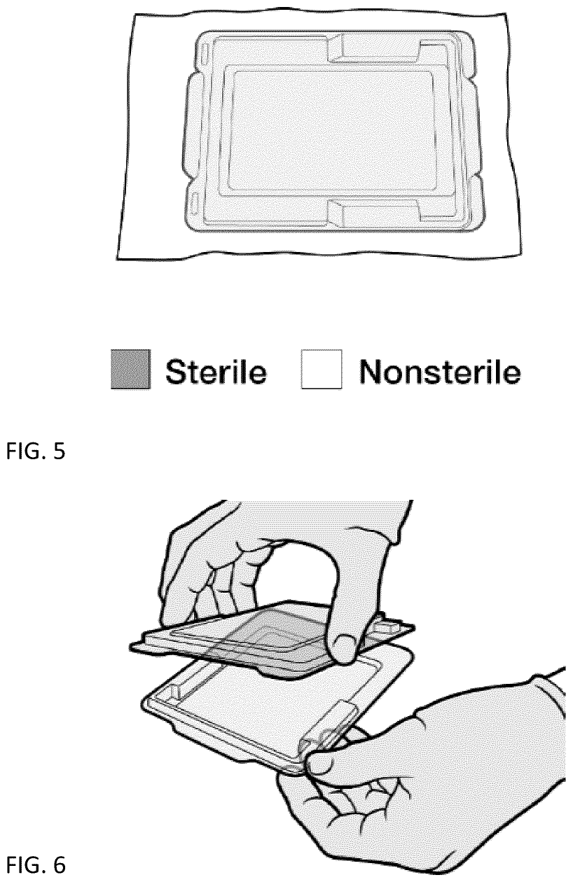

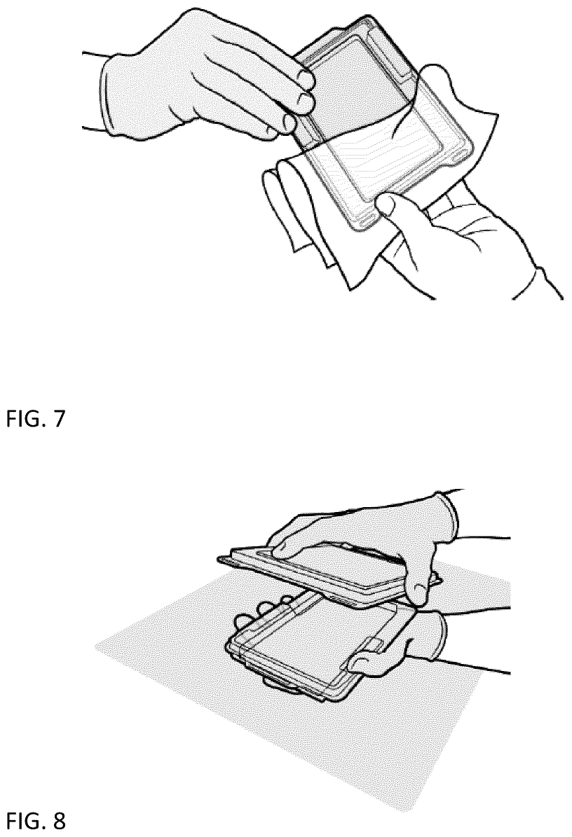

STRATAGRAFT is to be prepared in an appropriate surgical environment. STRATAGRAFT is supplied as follows: The STRATAGRAFT carton contains a laminated foil pouch (FIG. 1). Within the pouch is the STRATAGRAFT Product Dish that contains STRATAGRAFT on a polystyrene insert tray (FIG. 2). Inside the STRATAGRAFT Product Dish is the polystyrene insert tray that contains the STRATAGRAFT construct (FIG. 3). STRATAGRAFT is loosely adhered to a polycarbonate membrane contained within a polystyrene insert tray frame. Hold Solution is provided in a plastic bottle contained within a laminated, foil pouch (FIG. 4). The Hold Dish is contained in a clear pouch (FIG. 5) consisting of identical top and bottom portions (FIG. 6).

The following supplies are not included but needed for the preparation:
Mesher: STRATAGRAFT is compatible with autograft meshing devices (crushing or noncrushing) and can be meshed at ratios up to 1:1
Warming oven/cabinet or water bath

Following wound bed preparation (excision/debridement), determine the number of STRATAGRAFT constructs that need to be thawed. The following instructions are for preparing one STRATAGRAFT construct. Two operators (one sterile operator and one non-sterile operator) are required. If two or more STRATAGRAFT constructs are required, prepare them in one or more batches. STRATAGRAFT construct preparation should take approximately 20 minutes for a single construct or group of constructs. Preparation includes the following steps:
1. Remove the Hold Solution bottle from the laminated, foil pouch. The Hold Solution is a clear liquid. Do not use the Hold Solution if the contents of the bottle appear cloudy or turbid. Use one bottle of Hold Solution for one construct being prepared for placement.
2. Warm the Hold Solution in a warming oven/cabinet or a water bath.

### Option 1:

Place Hold Solution bottle in a warming oven/cabinet operating at 35° C. to 39° C. (95° F. to 102° F.) for AT LEAST 45 minutes prior to use.

### Option 2:

Place Hold Solution bottle in a water bath at 35° C. to 39° C. (95° F. to 102° F.) for AT LEAST 15 minutes prior to use. Do not submerge the cap or threads of the bottle.
3. The nonsterile operator peels open the seal of the Hold Dish pouch and aseptically presents the Hold Dish to the sterile operator for placement in the sterile field (FIG. 7). Sterile operator aseptically removes the Hold Dish presented by the nonsterile operator (FIG. 7) and places it in the sterile field (FIG. 8).
4. Remove STRATAGRAFT carton from the freezer or dry ice.

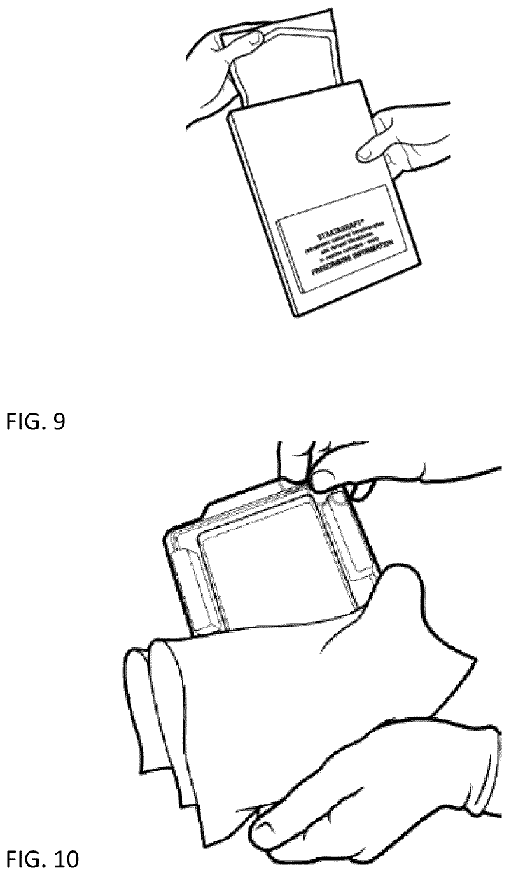

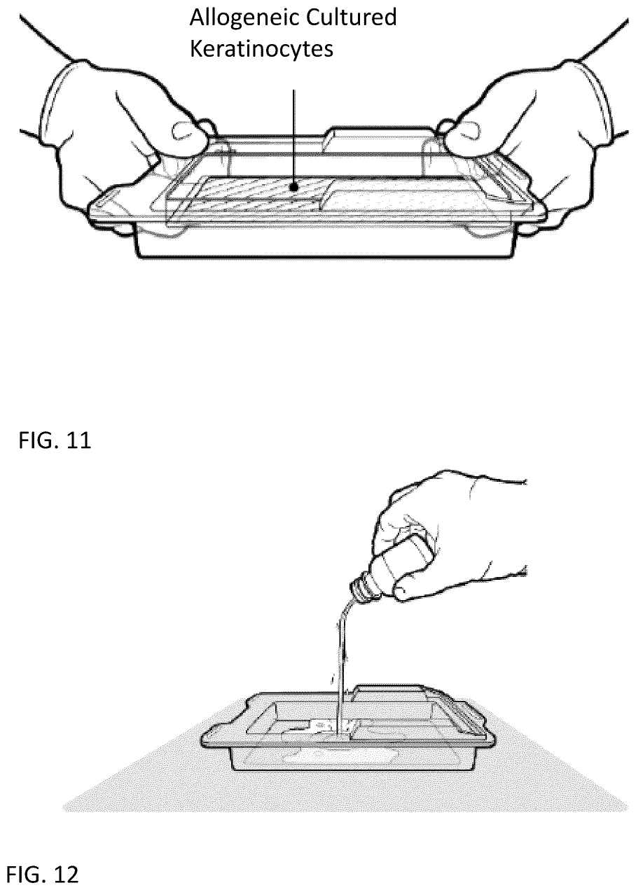

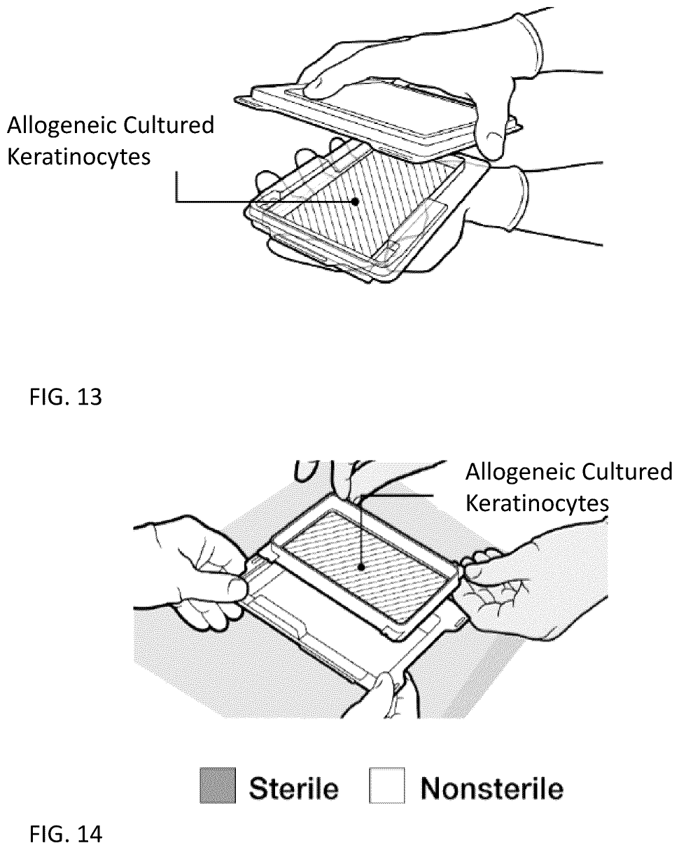

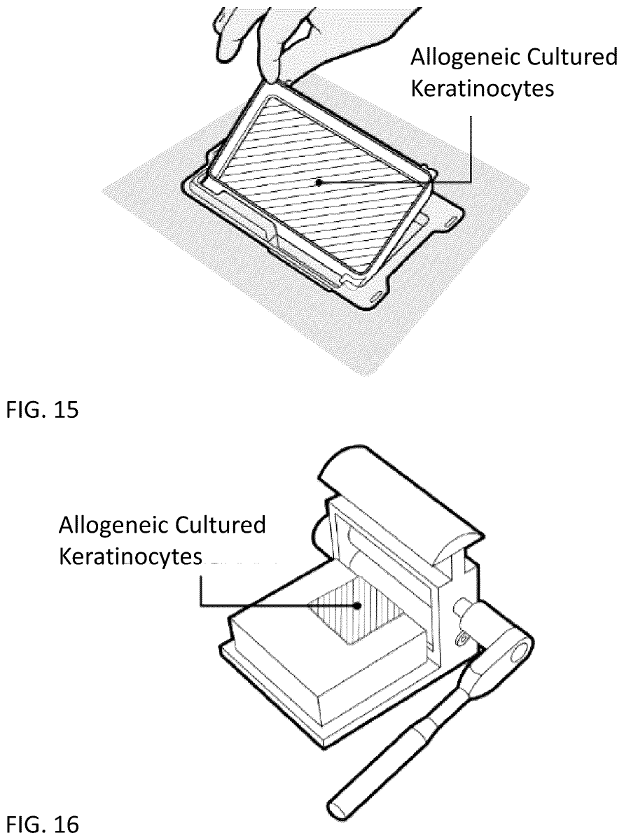

Note: Steps 4-11 must be completed within 10 minutes of removal of STRATAGRAFT from the freezer or dry ice.
5. Open the STRATAGRAFT carton and remove the foil pouch (FIG. 9).
6. Peel the foil pouch open and carefully remove the closed STRATAGRAFT Product Dish (FIG. 10).
7. The unopened STRATAGRAFT Product Dish containing the construct may be placed on a flat surface outside of the sterile field (FIG. 11).
8. The nonsterile operator pours one entire bottle of warmed Hold Solution into one sterile Hold Dish using aseptic technique per institutional procedures, immediately after removing the warmed Hold Solution from the warming device (FIG. 12).
9. The nonsterile operator removes the STRATAGRAFT Product Dish lid and holds the STRATAGRAFT Product Dish without contacting the insert tray (FIG. 13).
10. The sterile operator aseptically removes the STRATAGRAFT insert tray from the STRATAGRAFT Product Dish using either sterile forceps or sterile, gloved fingers (FIG. 14).
11. The sterile operator uses sterile, gloved fingers to place the insert tray into the Hold Dish beginning with one edge and lowering it to the opposite edge to minimize trapping bubbles beneath the insert tray (FIG. 15). If bubbles become trapped beneath the insert tray, gently lift the insert tray and place it slowly back down into the Hold Solution.
12. Maintain the STRATAGRAFT construct in the Hold Solution for a minimum of 15 minutes to a maximum of 4 hours.
13. Remove STRATAGRAFT from the polycarbonate membrane of the insert tray with sterile, gloved fingers or a pair of atraumatic forceps and mesh the STRATAGRAFT up to a 1:1 ratio. Do not allow STRATAGRAFT to dry. Moisten the mesher/tissue board as needed to prevent adhesion and maintain STRATAGRAFT moisture using Hold Solution, sterile 0.9% normal saline, or lactated Ringer's solution. STRATAGRAFT is packaged with the dermal (shiny) side in contact with the polycarbonate membrane. The construct may be meshed (FIG. 16) with either side up; note which side is the dermis (shiny) and which side is the epidermis (matte) prior to meshing and placement.

STRATAGRAFT is applied in appropriate aseptic conditions by a trained healthcare provider.

Follow the steps below for topical application of STRATAGRAFT:
1. If utilizing fibrin glue to anchor STRATAGRAFT, apply prior to placing the meshed STRATAGRAFT on the prepared wound bed. If utilizing anchoring methods other than fibrin glue, start with step 2.
2. Place the meshed STRATAGRAFT with the dermal (shiny) side down in contact with the patient's prepared wound bed. Ensure the epidermal (matte) side is facing up. About the constructs when placing multiple STRATAGRAFT constructs. Overlapping the STRATAGRAFT construct edges is not required.

Note: If the area to be treated is smaller than one construct, trim excess STRATAGRAFT

before or after anchoring to the wound bed. Ensure that STRATAGRAFT has contact across the entire surface of the wound bed. Do not stretch or expand STRATAGRAFT, as doing so may degrade the structural integrity of STRATAGRAFT.
3. Anchor STRATAGRAFT with staples, tissue adhesive such as cyanoacrylate, or sutures to keep STRATAGRAFT from dislodging. Place a porous, nonadherent contact dressing over STRATAGRAFT and leave the dressing in place for one week before changing.
4. Place a second layer of dressing that does not contain silver.
5. Placement of an outer bolster or wrap that keeps STRATAGRAFT from moving as clinically appropriate is at the discretion of the physician.

STRATAGRAFT is an off-white, rectangular sheet of approximately 100 cm² (approximately 8 cm by 12.5 cm) consisting of a viable, bioengineered, allogeneic cellularized scaffold product derived from keratinocytes grown on gelled collagen containing dermal fibroblasts.

Do not use in patients with known allergies to murine collagen or products containing ingredients of bovine or porcine origin.

STRATAGRAFT contains glycerin. Avoid glycerin in patients with known sensitivity (irritant reaction) to glycerin.

Severe hypersensitivity reactions may occur [see Contraindications (4)]. Monitor for both early and late symptoms and signs of hypersensitivity reaction following STRATAGRAFT application, and treat according to standard medical practice.

STRATAGRAFT contains cells from human donors and may transmit infectious diseases or infectious agents, e.g., viruses, bacteria, or other pathogens, including the agent that causes transmissible spongiform encephalopathy (TSE, also known as Creutzfeldt-Jakob disease (CJD) or variant CJD).

STRATAGRAFT is a xenotransplantation product because of an historic exposure of the keratinocyte cells to well-characterized mouse cells. The cell banks have been tested and found to be free of detectable adventitious agents and mouse cells are no longer used in the manufacture of STRATAGRAFT [see Description (11)]; however, these measures do not entirely eliminate the risk of transmitting infectious diseases and disease agents.

Transmission of infectious diseases or agents by STRATAGRAFT has not been reported.

Because STRATAGRAFT is a xenotransplantation product, STRATAGRAFT recipients should not donate whole blood, blood components, plasma, leukocytes, tissues, breast milk, ova, sperm, or other body parts for use in humans.

### Example 2: Clinical Studies

The efficacy of STRATAGRAFT in adult patients with thermal burns containing intact dermal elements for which surgical excision and autografting were clinically indicated was evaluated in two randomized, open label, intrapatient controlled, multicenter clinical studies of 12 months duration (Study 1 and Study 2). In both studies, two comparable wound sites of each patient were selected and randomized to receive either topical application of STRATAGRAFT or autograft. Autografts served as the intrapatient control.

Study 1 enrolled 71 adult patients with acute thermal burns containing intact dermal elements (deep partial-thickness burns) involving 3 to 37% total body surface area (TBSA). The time from burn to study treatment was 1 to 18 days. The size of the STRATAGRAFT-treated wound was 12 to 960 cm². The mean age was 44 years (19 to 79 years) with 78% male. Seventy-eight percent of patients were White, 20% were Black or African-American, and the remainder were Asian or Other. Efficacy was established on the basis of: (1) the difference in the percent area of the STRATAGRAFT treatment site and the control autograft treatment site that required autografting by 3 months following treatment, and (2) the proportion of patients achieving durable wound closure of the STRATAGRAFT treatment site at 3 months without autograft placement. Durable wound closure at 3 months was defined as wound closure at two consecutive study visits at least 2 weeks, but no more than 5 months apart, and including or encompassing the 3-month time point.

The difference in the percent area of STRATAGRAFT and control autograft treatment sites that required autografting by 3 months was 97.8%+16.6% (p<0.0001). Three patients had part or all of their STRATAGRAFT treatment site autografted by 3 months, and two of the three patients also had part or all of their autograft control study site re-grafted. Donor site harvest was eliminated for 96% (68/71) of STRATAGRAFT-treated burn sites. As a result, pain and scarring were significantly reduced at these potential donor sites that were spared and remained intact (p<0.0001). The cosmesis was similar between STRATAGRAFT treated sites and autograft treated sites.

The proportion of patients achieving durable closure of the STRATAGRAFT treatment site at 3 months without autograft placement was 83.1% (95% CI: 74.4, 91.8). The proportion of patients achieving durable closure of the autograft control treatment site at 3 months without additional autograft placement was 86% (95% CI: 77.8, 94.0).

In subgroups of race, ethnicity, sex, age, burn size in percentage of TBSA, STRATAGRAFT treatment area, and Baux scores (a scoring system to estimate mortality due to burn), efficacy results were in general consistent with the results in the overall Study 1 population.

Study 2 enrolled 30 adult patients with acute thermal burns containing intact dermal elements (deep partial-thickness burn) and involving 3 to 49% TBSA. The time from burn to study treatment ranged from 3 to 13 days. The size of the STRATAGRAFT-treated wound was 52 to 440 cm². The mean age was 41 years (21-63 years). Ninety-three percent of patients were White and 7% were Black or African-American. Men accounted for 70% of the population.

Efficacy was evaluated on the basis of: (1) the percent area of STRATAGRAFT treatment site autografted by 28 days after STRATAGRAFT treatment, and (2) the proportion of treatment sites that achieved complete wound closure by 3 months. Complete wound closure was defined as >95% re-epithelialization in the absence of drainage.

No STRATAGRAFT treatment site required autografts by 28 days. Between 28 days and 3 months, one patient had both the STRATAGRAFT treatment site and the autograft site treated subsequently with autograft, and a second patient had 25% of the STRATAGRAFT treatment site autografted. At 3 months, 93.1% of STRATAGRAFT treatment sites and 100% of autograft treatment sites achieved complete wound closure. All STRATAGRAFT treatment sites that achieved complete wound closure at 3 months remained closed when evaluated at 6 months and 12 months after treatment.

### Example 3: Clinical Pharmacology

STRATAGRAFT is an allogeneic cellularized scaffold product containing metabolically active cells that produce and secrete a variety of growth factors and cytokines. In vitro studies have shown that STRATAGRAFT secretes human growth factors and cytokines, and contains human extracellular matrix proteins. Growth factors, cytokines, and extracellular matrix proteins are known to be involved in wound repair and regeneration.

STRATAGRAFT does not remain permanently engrafted, but is replaced by the patient's own cells over time, eliminating or reducing the need for autografting to attain definitive closure of the majority of treated wounds. A total of 85 patients in Study 1 and Study 2 were evaluated at 3 months for persistence of allogeneic STRATAGRAFT DNA at the treatment site. Specifically, at Month 3, tissue samples from the STRATAGRAFT treatment sites were tested for the presence of allogeneic DNA from STRATAGRAFT and compared with results from blood reference samples from baseline. STRATAGRAFT-associated DNA was not detected in these patients. The analysis showed that no patients (n=57) displayed signs of residual allogeneic DNA from STRATAGRAFT at STRATAGRAFT treatment sites at Month 3, which is consistent with wound healing by the patients' own cells.

The pharmacodynamics and pharmacokinetic effects of STRATAGRAFT are not known.

### Example 4: Adverse Reactions and Use in Specific Populations

The most common adverse reactions (incidence 22%) were itching (pruritus), blisters, hypertrophic scar and impaired healing. (Table 1).

Because clinical trials are conducted under widely varying conditions, adverse reaction rates observed in the clinical trials of a drug cannot be directly compared to rates in the clinical trials of another drug and may not reflect the rates observed in practice. The safety data described in this section reflect exposure to STRATAGRAFT in four randomized, within-subject controlled studies conducted in the United States, including two clinical studies in patients with deep partial-thickness thermal burns (Study 1 and Study 2) and two clinical studies in patients with full-thickness complex skin defects due to thermal burns or other causes. A total of 119 adult patients (101 patients with deep partial-thickness thermal burn, and 18 patients with full-thickness complex skin defects) received topical application of STRATAGRAFT. The patient population ranged in age from 19 to 79 years (mean age 43 years). Each patient received topical application of STRATAGRAFT at one wound site and either autografting (104 patients) or cadaver allografts (15 patients) as intrapatient comparators at another wound site. The most frequent adverse reactions (incidence 22%) observed in the 4 studies are summarized in Table 1. No patients discontinued study participation due to adverse reactions.

Overall, the safety profile of STRATAGRAFT with regard to wound-related events, including erythema, swelling, local warmth and wound site infections, was similar to that of autografting in these studies.

There were no reports of rejection to STRATAGRAFT in the clinical studies. The safety of STRATAGRAFT beyond 12 months was not evaluated in the clinical studies.

TABLE 1
Adverse Reactions Reported at a Frequency of ≥2%
Following Treatment with STRATAGRAFT (N = 119)

| | Patients |
|---|---|
| Adverse Reaction | n (%)* |
| Pruritus (Itching) | 13 (11) |
| Blisters | 5 (4) |
| Hypertrophic Scar | 3 (3) |
| Impaired Healing | 3 (3) |

Potential Drug Interactions were evaluated as well. The use of mafenide acetate is not recommended following application of STRATAGRAFT. This topical antimicrobial has been shown to reduce keratinocyte viability and disrupt the integrity of tissue-engineered human skin substitutes composed of NIKS® keratinocytes or primary keratinocytes (See Gibson A L, et al. Tissue Eng Part A. 2008 May: 14 (5): 629-38).

The use of silver-containing antimicrobials or dressings is not recommended because in vitro data suggest silver may decrease the viability of keratinocytes and human dermal fibro-blasts (See Nešporová K, et al. Scientific Reports. 2020 September; 10(1): 15216).

The use of antimicrobial chlorhexidine solution on the wound following application of STRATAGRAFT is not recommended. This material has been shown to be toxic to keratinocytes and human dermal fibroblasts (See Boyce S T, et al. J Burn Care Rehabil. 1995 March-April; 16 (2 Pt 1): 97-103).

### Example 5

Following placement in an excised wound bed, StrataGraft skin tissue may be secured using a variety of fixation methods routinely used to affix skin grafts. Fibrin sealants have been investigated as an alternative to traditional fixation methods (i.e. sutures or staples) due to their quick application process and improved wound bed adherence¹. The use of fibrin glue has also been shown to improve the percentage of skin graft take; this is especially true when the burn injury is located in an area that is difficult to graft or immobilize². In addition, evidence suggests that the adhesive properties of fibrin sealants may reduce the occurrence of bacterial infection, blood loss, and postoperative pain^{2,3}. Because StrataGraft tissues contain viable cells that secrete a variety of wound healing factors, it is possible that sealants that negatively impact the tissue viability may reduce the efficacy of the product.

Fixation of Stratagraft with fibrin glue has not been previously evaluated in experimental models or clinical studies. In order to establish the suitability of fibrin glues for use with StrataGraft, this study was performed to assess the impact of ARTISS fibrin sealant on tissue viability.

The results of this study showed that exposure to ARTISS fibrin glue does not negatively impact tissue viability. Interestingly, the experimental group actually saw a statistically significant increase in viability compared to controls, a result that was not observed in two pilot studies. These results indicate that the use of ARTISS with StrataGraft skin tissue does not result in substantial cell death, making it unlikely that treatment outcomes will be compromised.

### Experimental Design:

The suitability of fibrin glue was assessed using Stratagraft tissue and nonviable simplified dermal equivalents (sDEs).

These studies made use of an in vitro wound model system consisting of tissue punches cultured on nonviable sDE layers produced in 6-well format tissue culture inserts. The nonviable sDEs were generated in the Stratatech research lab using a modified batch record developed specifically for this project. At the end of production, the sDEs were aseptically transferred within 6-well inserts into sterile 6-well dishes, and then sealed inside final product pouches using standard procedures. Following the final product packaging step, sDEs were frozen in an ultracold freezer and stored at −70 to-90° C. until thawed for use. Because there was no cryoprotectant treatment step, freezing the sDE eliminates the viability of the normal human dermal fibroblasts (NHDF). These nonviable sDEs provide a dermal matrix on which samples of StrataGraft tissue were placed during the fibrin glue interaction studies.

On the day of the thaw (day 0), reculture chambers were assembled by placing sterile mesh lifters in p150 dishes and adding 50 mL of buffer to each dish, sDEs were thawed for 3 to 5 minutes at ambient temperature and subsequently transferred within their 6-well inserts to the reculture dishes and placed on top of the mesh lifters. StrataGraft tissues were thawed for 3 to 6 minutes at ambient temperature, and 15 mL of Stratatech hold solution (warmed NLT 60 minutes in a 35-39° C. water bath or dry block) was added to a new product dish for each tissue being thawed. Tissues were held for 15 to 20 minutes at ambient temperature. ARTISS fibrin sealant was applied to the sDEs as indicated in Table 2, with care being taken to disperse a thin, even layer onto each dermis. Each condition was tested in quintuplicate. After the post-thaw hold step, 8 mm punches were taken from the tissue, and each punch was transferred to a reculture dish and placed dermis side down onto the sealant layer. The sDE/tissue punch assemblies were transferred to a humidified incubator.

TABLE 2
Experimental

| Group | Volume of Sealant Applied |
|---|---|
| A (Control) | N/A |
| B | 5 drops |

Media exchanges were performed on days 1 and 4. After 5 total days of contact time, the tissue punches were analyzed for the StrataGraft stability-indicating assay of viability. The results of the punches exposed to the fibrin glue were compared to the results from the control group. Fibrin glue exposure resulting in viability reductions of <10% is considered to have a negligible effect on the tissue, 10-30% is considered a moderate effect, and reductions of >30% are considered a substantial effect.

Statistical analysis of the viability results was performed to determine the effects of each condition. One-way ANOVA tests were constructed for the fibrin sealant group to compare the results of exposure to the control group. Statistical significance is defined as p<0.05.

### Results:

A summary of the analysis results is presented below, and the complete dataset is provided in Table 3. The effects of exposing tissues to ARTISS fibrin sealant were assessed.

### Viability:

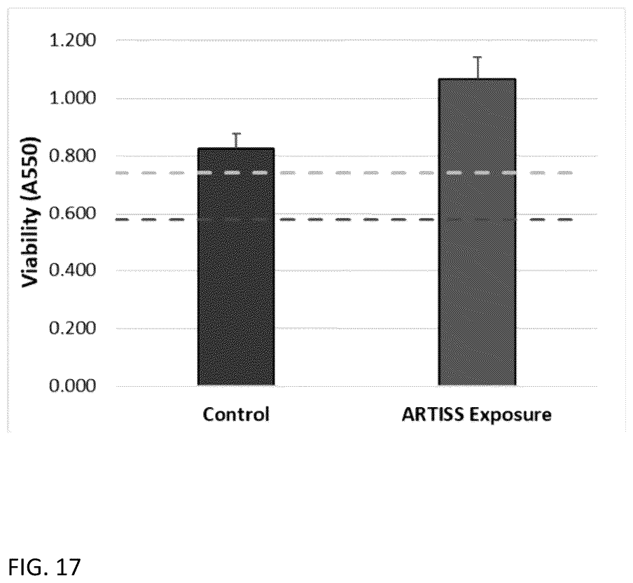

Exposure to ARTISS fibrin glue was not found to have a negative impact on tissue viability when compared to the control group. The data are presented in FIG. 17. Analysis showed a significant effect of fibrin sealant exposure on the resulting sample viability (p<0.001), with the experimental group showing a 30% increase in viability over the control.

Although these results strongly support the position that the use of ARTISS does not negatively impact the StrataGraft tissue, the reason for the increase in measured viability was not clear. It could be the result of increased proliferation and migration of NHDF and/or NIKS cells from the tissue punch, but this seems unlikely based on the staining patterns. Post-viability images show that samples in both the control and ARTISS groups had robust MTT staining of the tissue punches, but also showed diffuse staining in sDE regions away from the punch. The models exposed to ARTISS appeared to have more of this visible pigmentation in the sDE region when compared to the control models, which could indicate the presence of viable cells. Although it would seem unlikely, the staining in the sDE layer could suggest some limited survival of cells during the freeze/thaw cycle meant to devitalize that layer. In that instance, the difference between the groups may reflect some protective effect of the fibrin glue on those residual cells.

### Complete Data Summary

TABLE 3
Complete Viability Data

| Model | | 1 | 2 | 3 | 4 | 5 | Average |
|---|---|---|---|---|---|---|---|
| Group | Control | 0.757 | 0.839 | 0.775 | 0.854 | 0.883 | 0.822 |
| | ARTISS | 1.151 | 1.044 | 1.020 | 1.131 | 0.980 | 1.065 |
| | Exposure | | | | | | |

### Discussion:

To help with interpretation of these results, we also considered the outcomes of earlier studies done to establish the model parameters. Prior to the performance of this study, two pilot experiments were performed to determine the volume of sealant that should be used with the models as well as to allow the operator to become proficient at the application method. These studies followed the same procedure outlined in the Experimental Design section with the exception of the volume of ARTISS applied in the first pilot study. As can be seen in Table 4, neither pilot study showed statistically significant effects of ARTISS exposure on tissue viability; one study showed modest, non-significant increases in viability with ARTISS usage (pilot study 1: p=0.541), while the second study showed small, non-significant decreases (pilot study 2: p=0.274). It is worth noting that the staining in the sDE region that was seen in this study was not seen in either pilot experiment. While the presence of this staining cannot be fully explained, taken together, the results of the pilot and formal studies indicate that ARTISS does not negatively affect tissue viability.

TABLE 4
Preliminary Runs Viability Data

| Pilot Study | Model | | 1 | 2 | 3 | 4 | Average |
|---|---|---|---|---|---|---|---|
| 1 | Group | Control | 0.817 | 0.682 | 0.760 | 0.786 | 0.761 |
| | | ARTISS Exposure- 1 | 0.735 | 0.747 | 0.830 | 0.799 | 0.778 |
| | | ARTISS Exposure- 3 | 0.857 | 0.835 | 0.743 | 0.777 | 0.803 |
| 2 | Group | Control | 0.760 | 0.663 | 0.635 | 0.748 | 0.702 |
| | | ARTISS Exposure- 3 | 0.630 | 0.163 1 | 0.673 | 0.642 | 0.648 |
| | | ARTISS Exposure- 5 | 0.679 | 0.724 | 0.677 | 0.689 | 0.692 |

1 The tissue punch in this model was inadvertently recultured dermis side up. As a result, this data point was excluded from the average for this group.

### Conclusions:

Based on the results of this experiment as well as those seen in the pilot studies, it can be concluded that the use of ARTISS fibrin sealant with StrataGraft skin tissue does not result in substantial cell death. Interestingly, the models exposed to the fibrin glue in this study saw a statistically significant, increased viability compared to the control group. These results indicate that it is unlikely that the use of ARTISS would compromise StrataGraft treatment outcomes.

### Example 6: Elemental Analysis

For the extractable study, the Growth Chamber and Tissue Insert assembly were extracted using 20% ethanol/80% water and the extracts were pooled and transferred to a 50-mL plastic volumetric flask. Similarly, for the culture media leachable study, the test materials were digested using the CEM Discover SPD Microwave Digestion System and the digested samples were transferred to a 50-mL plastic volumetric flask.

To this volumetric flask, an internal standard containing Beryllium (Be), Germanium (Ge), Indium (In), Terbium (Tb), and Scandium (Sc) were added to a final concentration of 50 ng/ml. The flask was filled to volume with 5% nitric acid. External standards were prepared at elemental concentrations ranging from 0.5-100 ng/ml. The standards contained each of the following elements: Lithium (Li), Boron (B), Magnesium (Mg), Aluminum (Al), Titanium (Ti), Vanadium (V), Chromium (Cr), Manganese (Mn), Iron (Fe), Cobalt (Co), Nickel (Ni), Copper (Cu), Zinc (Zn), Arsenic (As), Selenium (Se), Molybdenum (Mo), Cadmium (Cd), Tin (Sn), Antimony (Sb), Barium (Ba), Mercury (Hg), Lead (Pb). The internal standards, consisting of Beryllium (Be), Scandium (Sc), Germanium (Ge), Indium (In), and Terbium (Tb), were added to these standards at a final concentration of 50 ng/mL. The content of each of these elements in the extraction samples were determined against the standards. As part of the system suitability requirements for the assay, the percent recovery of the recovery surrogate compounds must be 80 to 120%. The results of the study are provided in Table 5.

TABLE 5
Method Performance (Surrogate Recoveries)
using the ICP-MS Method

| | Coefficient of | System | QC Standard |
|---|---|---|---|
| | Linearity | Precision | % Recovery |
| Specification | ≥0.995 | % RSD ≤10% | 80 to 120% |
| Cd | 1.000 | 1 | 101 |
| Pb | 1.000 | 2 | 96 |
| As | 1.000 | 4 | 105 |
| Hg | 1.000 | 1 | 96 |
| Co | 1.000 | 2 | 99 |
| V | 1.000 | 3 | 104 |
| Ni | 1.000 | 1 | 101 |
| Se | 1.000 | 3 | 94 |
| Li | 1.000 | 2 | 109 |
| Sb | 1.000 | 1 | 99 |
| Ba | 1.000 | 1 | 101 |
| Mo | 1.000 | 4 | 102 |
| Cu | 1.000 | 2 | 102 |
| Sn | 1.000 | 2 | 102 |
| Cr | 1.000 | 1 | 100 |
| Al | 0.999 | 1 | 107 |
| B | 1.000 | 2 | 106 |
| Ti | 1.000 | 5 | 95 |
| Mn | a1.000 | 1 | 97 |
| | Internal Standards | | |
| | (Specification: 80 to 120%) | | |
| | Be | Ge | In |
| % Recovery | 95 | 100 | 99 |

### Results of Growth Chamber and Tissue Insert Assembly Studies

When assessing the extractable and leachable elemental impurities using the ICP-MS method, most elements were either not detected (ND) or below the LOQ (Limit of Quantitation) for the method. If the response for an element in the sample is less than or equal to the response for that element in the control, then the result reported is not detected (ND). The results of the analysis are provided in Table 6. Boron (B) and aluminum (Al) were detected at sub-ppm range in the extractable study, however only Boron was detected in the leachable study. Tin was detected at low level in one replicate during the leachable study, however the detected level of tin does not pose a risk to patients receiving a dosage of 30 StrataGraft skin tissues as the levels are below the permitted daily allowance limits.

TABLE 6
Summary of Elemental Impurities Detected by ICP-MS

| | 20% Ethanol Extractables Results (ppm) | | | SM Media Leachables Results (ppm) | | |
|---|---|---|---|---|---|---|
| Element | Collection 1 | Collection 2 | Collection 3 | Collection 1 | Collection 2 | Collection 3 |
| Li | ND | ND | ND | ND | ND | ND |
| B | 0.45 | 0.48 | 0.5 | 0.13 | <LOQ | 0.23 |
| Al | 0.82 | 0.48 | 0.59 | <LOQ | ND | ND |
| Ti | ND | ND | ND | ND | ND | ND |
| V | ND | ND | ND | <LOQ | <LOQ | <LOQ |
| Cr | ND | ND | <LOQ | <LOQ | <LOQ | <LOQ |
| Mn | ND | ND | ND | ND | ND | ND |
| Co | ND | <LOQ | ND | ND | ND | <LOQ |
| Ni | ND | ND | ND | ND | ND | <LOQ |
| Cu | <LOQ | ND | ND | <LOQ | <LOQ | <LOQ |
| As | ND | <LOQ | <LOQ | ND | ND | <LOQ |
| Se | ND | <LOQ | ND | ND | ND | ND |
| Mo | ND | ND | <LOQ | ND | ND | <LOQ |
| Ru | ND | ND | ND | <LOQ | ND | ND |
| Rh | ND | ND | ND | <LOQ | <LOQ | <LOQ |
| Ag | ND | ND | ND | 0.54 | ND | ND |
| Cd | ND | ND | ND | <LOQ | <LOQ | <LOQ |
| Sn | ND | ND | ND | ND | ND | 0.13 |
| Sb | ND | ND | ND | <LOQ | ND | <LOQ |
| Ba | <LOQ | ND | ND | <LOQ | ND | <LOQ |
| Hg | ND | ND | ND | ND | ND | ND |
| Tl | ND | ND | ND | ND | ND | ND |
| Pb | ND | ND | ND | ND | ND | ND |

ND = Not Detected
<LOQ = Below Limit of Quantitation

### Product Dish/Hold Dish

The extracts used for ICP/MS analysis were prepared separately. The extraction was performed with pH 3 water and pH 9 water. 18 mL of the aforementioned extraction solvent was added into the Product Dish and the Product Dishes were incubated at 37±1° C. for 24 hours. The extraction was performed on three Product Dishes. After the incubation, the extracts were pooled in a volumetric flask resulting in one replicate for each condition. An internal standard containing Beryllium (Be), Germanium (Ge), Indium (In), Terbium (Tb), and Scandium (Sc) were added to each volumetric flask to achieve a final concentration of 50 ng/ml. The flask was filled to volume with 5% nitric acid.

External standards were prepared at elemental concentrations ranging from 0.5-100 ng/ml. The standards contained each of the following elements: Lithium (Li), Boron (B), Aluminum (Al), Titanium (Ti), Vanadium (V), Chromium (Cr), Manganese (Mn), Cobalt (Co), Nickel (Ni), Copper (Cu), Arsenic (As), Selenium (Se), Molybdenum (Mo), Cadmium (Cd), Tin (Sn), Antimony (Sb), Barium (Ba), Mercury (Hg), and Lead (Pb). The internal standard was also added to these standards at a final concentration of 50 ng/ml. The content of each of these elements in the extraction samples was determined against the standards (Table 17).

TABLE 17
Method Performance (Surrogate Recoveries)
using the ICP-MS Method

| | Coefficient | System | QC Standard % Recovery | | |
|---|---|---|---|---|---|
| | of linearity | Precision | Q1 | Q2 | Q3 |
| Specification | ≥0.995 | % RSD | 80 to | 80 to | 80 to |
| | | S 10% | 120% | 120% | 120% |
| Cd | 1.000 | 3.8 | 100 | 95 | 100 |
| Pb | 1.000 | 3 | 102 | 102 | 103 |
| As | 1.000 | 9.1 | 97 | 102 | 100 |
| Hg | 1.000 | 8.1 | 108 | 101 | 92 |
| Co | 1,000 | 3.9 | 101 | 105 | 101 |
| V | 1.000 | 2.3 | 104 | 108 | 103 |
| Ni | 0.999 | 5.2 | 89 | 81 | 85 |
| Se | 0.998 | 6.2 | 104 | 120 | 90 |
| Li | 1.000 | 1.5 | 101 | 97 | 97 |
| Sb | 1.000 | 4.3 | 104 | 105 | 103 |
| Ba | 0.999 | 8.2 | 111 | 112 | 113 |
| Mo | 1.000 | 5 | 100 | 100 | 96 |
| Cu | 1.000 | 2.1 | 100 | 103 | 102 |
| Sn | 1,000 | 6.2 | 100 | 100 | 102 |
| Cr | 1.000 | 2.2 | 103 | 105 | 100 |
| Al | 1.000 | 1 | 110 | 117 | 115 |
| B | 1.000 | 6.3 | 103 | 94 | 99 |
| Ti | 0.998 | 9.3 | 107 | 112 | 118 |
| Mn | 1.000 | 2.6 | 109 | 115 | 110 |
| W | 1.000 | 5.7 | 105 | 96 | 96 |
| | Internal Standard | | | | |
| | Be | Sc | Ge | In | Tb |
| % Recovery | 97 | 101 | 104 | 104 | 102 |
| (Run 1) | | | | | |
| % Recovery | 105 | 103 | 104 | 105 | 105 |
| (Run 2) | | | | | |

The Target elements for the ICP-MS analysis were chosen based on USP<232> and ICH Q3D guidelines. Their classification of three classes and their corresponding Permitted Daily Exposure Limits (PDEs) were recommended in the ICH Q3D guideline. Most of the elements were either not detected (ND) or below LOQ as provided in Table 7. If the response for an element in the sample is less than or equal to the response in the control, then the result reported is non-detected (ND). Sn was detected but well below its respective limit.

TABLE 7
ICP-MS Analysis of Extraction Samples

| | | Parenteral Limit per ICHQ3D | | | Results (ng/mL = ppb) | |
|---|---|---|---|---|---|---|
| | | PDE | Concentration | Concentration | Water | Water |
| Element | Class | (μg/day) | Limit (μg/mL)¹ | Limit (ng/mL)² | pH 9 | pH3 |
| Cd | 1 | 2 | 0.004 | 4 | <LOQ | ND |
| Pb | 1 | 5 | 0.009 | 9 | ND | ND |
| As | 1 | 15 | 0.028 | 28 | ND | ND |
| Hg | 1 | 3 | 0.006 | 6 | ND | ND |
| Co | 2A | 5 | 0.009 | 9 | ND | ND |
| V | 2A | 10 | 0.019 | 19 | ND | <LOQ |
| Ni | 2A | 20 | 0.037 | 37 | ND | <LOQ |
| Se | 2B | 80 | 0.148 | 148 | <LOQ | ND |
| Li | 3 | 250 | 0.463 | 463 | ND | ND |
| Sb | 3 | 90 | 0.167 | 167 | <LOQ | <LOQ |
| Ba | 3 | 700 | 1.296 | 1296 | <LOQ | ND |
| Mo | 3 | 1500 | 2.778 | 2778 | <LOQ | ND |
| Cu | 3 | 300 | 0.556 | 556 | ND | ND |
| Sn | 3 | 600 | 1.111 | 1111 | 0.24 | 2.71 |
| Cr | 3 | 1100 | 2.037 | 2037 | ND | <LOQ |
| B | NA | NA | NA | NA | 0.31 | 0.26 |
| Al | NA | NA | NA | NA | 1.97 | 2.24 |
| Ti | NA | NA | NA | NA | ND | <LOQ |
| W | NA | NA | NA | NA | ND | ND |

LOQ = limit of quantitation; N/A = not applicable; ND = not detected.
¹Permitted Daily Exposure (PDE) limits for parenteral products based on ICHQ3D guidelines.
²Thirty tissues per day and a volume of 18 mL extracting solvent per Product Dish (or Hold Dish) were applied to determine each element impurity concentration limit in μg/mL. Cd example: 2 μg/day X day/30 tissues X bottle/18 mL = 0.004 μg/mL (ppm) = 4 ng/mL (ppb)

B and Al were detected at low ppb levels. For aluminum, WHO has a recommendation not to exceed 1.8 mg/day for an adult from drinking water. As such, the ppb level of aluminum is negligible, does not pose a risk to patients. Boron is a probably essential element to human body by WHO. US EPA has a limit to drinking water of boron at 3 mg/L (assuming children consuming 1 L/per day, while adult consuming 2 L/per day), as such, the daily safety level of boron intake is between 3 mg to 6 mg per day. Therefore, the boron at ppb level in the leachable study is negligible, it does not pose any risk to our patients.

The scope of ICH Q3D (R1) Guideline for Elemental Impurities does not apply to tissue engineering products such as StrataGraft skin tissue. However, the extractable and leachable (E&L) assessment for StrataGraft skin tissue has taken into account the potential contribution of elemental impurities from the plastic manufacturing components used to culture the skin tissue and the primary packaging components used to cryopreserve and store the tissue until clinical application. The manufacturing process of StrataGraft skin tissue incorporates multiple medium exchanges during the 25 day organotypic culture period. There is no scientific evidence that trace amounts of elemental impurities introduced from the manufacturing plasticware (namely the Growth Chamber and Transwell Insert) to the culture medium would accumulate within the cellular components of StrataGraft skin tissue. Thus, it can be hypothesized that elemental impurities introduced from the manufacturing plasticware would be diluted or removed by way of the medium exchanges during the tissue manufacturing process. Similarly, there is no scientific evidence that the elemental impurities introduced from the primary packaging components would accumulate within the finished drug product. Prior to clinical application, the skin tissue is rinsed in StrataGraft Hold Solution. Thus, the preparation of the tissue for clinical application may dilute or remove these impurities from the skin tissue. The elemental impurities data provided in this study are compared against the ICH Q3D limits due to a lack of alternate standards and are meant for informational purposes. The risk assessment for the elemental impurities suggest that the risk of delivery of the full volume of impurities to a patient, as being provided in this study, is minimal based on tissue manufacturing and clinical preparation procedures.

### Leachable Study

This study considers the potential for leachables from the Product Dish and Hold Dish. The Product Dish and Hold Dish are identical components; however, each of the dishes are exposed to different medium and environmental conditions during long-term storage and clinical use, respectively. The Product Dish is stored at −70 to −90° C. throughout the shelf life of the drug product. Prior to use, Hold Solution bottles are pre-warmed to 35 to 39° C. and then poured into sterile Hold Dishes. During the hold period, a portion of the Hold Solution moves into the tissue, displacing the cryoprotectant and maintaining the tissue hydration. Once the tissue is removed from the Hold Dish for clinical use, the described portion of the volume of Hold Solution is delivered, as part of the product, to the patient upon tissue application. The exposure period of the Hold Dish to the pre-warmed Hold Solution is at least 15 minutes and up to 4 hours.

### Product Dish

For the evaluation of the Product Dish, 3 mL of CPS was added to each Product Dish. The 3 mL CPS exposure approximates the maximum amount of CPS expected to be transferred to the Product Dish via adherence to the Tissue Insert during transfer of the StrataGraft skin tissue to the Product Dish. After adding the CPS, the Product Dish was sealed in a foil pouch and held for 30 minutes at room temperature. The pouches containing the Product Dish were then transferred to an ultra-cold freezer for long-term storage at −70 to −90° C. In order to differentiate CPS-related components from leachable compounds, time zero (TO) and control samples consisted of CPS that was stored in sterilized Teflon bottles. The evaluation of leachables will be performed at T0, and 3-, 6-, 12-, 18-, 24-, and 36-month time points.

Cramer class III guidelines were used in this study to define the AET, which will allow up to 90 jig of an unknown compound per application of StrataGraft skin tissues. For the purposes of the extractable leachable studies, a maximum application of up to 30 tissues per day were factored into the calculation. Thus, the AET would be set to 3 jig per tissue or 3 jig per Product Dish.

### Hold Dish

A one-time study was performed for the evaluation of the Hold Dish leachables to simulate clinical-use conditions. Three mL of CPS and 15 mL of Hold Solution were mixed before adding to each Hold Dish. The Hold Dishes containing the mixed medium were conditioned at 37° C. for 4 hours; the maximum allowed duration of the clinical hold. The experiment was performed in duplicate with three Hold Dishes for each replicate. After conditioning, the test solution consisted of material pooled from the three Hold Dishes. The controls were prepared the same way as the test samples. Three mL of CPS and 15 mL of Hold Solution were combined in a glass bottle and conditioned at 37° C. for 4 hours.

A conservative estimate of the volume of the CPS and Hold Solution mixture, which moves into the tissue, was used to calculate the total amount of a leachable per Hold Dish that might be delivered to the patient. The fluid volume of a StrataGraft skin tissue should not exceed 2.5 mL, so a maximum of 2.5 mL from the total 18 mL in the Hold Dish would be contained in the tissue applied to a patient. Therefore, the potential exposure from the Hold Dish is calculated as:

$$\begin{matrix}Concentration from the 18 mL of \\ CPS and Hold Solution ( μg / mL )\end{matrix} \times \begin{matrix}2.5 mL per tissue = Total \\ leachable per Hold Dish ( μg / dish )\end{matrix}$$

The results from leachables testing of the Hold Dish containing combined CPS and Hold Solution are combined with the results from the leachables study evaluating long term storage of the drug product in CPS in the Product Dish. For example, if a leachable compound is detected in both the Hold Dish study and the long-term storage Product Dish leachables study, the total exposure will be calculated as the sum of the leachable from one Product Dish and one Hold Dish. That total is compared to the 3 μg/dish AET.

### Preparation of Leachate Samples for ICP-MS Analysis

For the culture media leachable study, the test materials were digested using the CEM Discover SPD Microwave Digestion System and the digested samples were transferred to a 50-mL plastic volumetric flask. To this flask, an internal standard containing Beryllium (Be), Germanium (Ge), Indium (In), Terbium (Tb), and Scandium (Sc) was added to a final concentration of 50 ng/ml. The flask was filled to volume with 5% nitric acid.

External standards were prepared at elemental concentrations ranging from 0.5-100 ng/ml. The standards contained each of the following elements: Lithium (Li), Boron (B), Aluminum (Al), Titanium (Ti), Vanadium (V), Chromium (Cr), Manganese (Mn), Cobalt (Co), Nickel (Ni), Copper (Cu), Zinc (Zn), Arsenic (As), Selenium (Se), Molybdenum (Mo), Cadmium (Cd), Tin (Sn), Antimony (Sb), Barium (Ba). Tungsten (W), Mercury (Hg), and Lead (Pb). Magnesium (Mg) and Iron (Fe) were excluded from the study due to their presence in the leachate samples from the compounds used to formulate the CPS and Hold Solution. The internal standards, consisting of Beryllium (Be), Scandium (Sc), Germanium (Ge), Indium (In), and Terbium (Tb), were added to these standards at a final concentration of 50 ng/ml. The content of each of these elements in the extraction samples were determined against the standards. As part of the system suitability requirements for the assay, the percent recovery of the recovery surrogate compounds must be 80 to 120%. The results of the study are provided in Table 8.

TABLE 8
Method Performance (Surrogate Recoveries)
using the ICP-MS Method

| | | | QC Standard | | |
|---|---|---|---|---|---|
| | Coefficient of | System | % Recovery | | |
| | linearity | Precision | Q1 | Q2 | |
| Specification | ≥0.995 | % RSD ≤10% | 80 to | 80 to | 80 to |
| | (except Al is | (1 ppb std, | 120% | 120% | 120% |
| | ≥0.985) | n = 6) | | | |
| Element | | | | | |
| Cd | 1.000 | 2 | 101 | 102 | 100 |
| Pb | 1.000 | 1 | 100 | 99 | 98 |
| As | 1.000 | 6 | 99 | 100 | 97 |
| Hg | 1.000 | 6 | 102 | 101 | 101 |
| Co | 1.000 | 4 | 99 | 95 | 95 |
| V | 1.000 | 5 | 99 | 99 | 98 |
| Ni | 1.000 | 3 | 89 | 83 | 82 |
| Se | 0.999 | 7 | 107 | 101 | 104 |
| Li | 0.999 | 3 | 107 | 109 | 107 |
| Sb | 1.000 | 4 | 99 | 100 | 99 |
| Ba | 1.000 | 7 | 103 | 98 | 96 |
| Mo | 1.000 | 4 | 95 | 95 | 99 |
| Cu | 1.000 | 3 | 109 | 88 | 90 |
| Sn | 1.000 | 3 | 97 | 90 | 93 |
| Cr | 1.000 | 5 | 91 | 93 | 90 |
| Al | 0.999 | 2 | 106 | 102 | 100 |
| B | 1.000 | 7 | 99 | 102 | 100 |
| Ti | 0.999 | 4 | 100 | 104 | 102 |
| Mn | 0.999 | 4 | 91 | 93 | 90 |
| W | 0.999 | 3 | 112 | 114 | 115 |

Internal Standard % Recovery
(Specification: 75 to 125%)

| | Sc | Ge | In | Tb |
|---|---|---|---|---|
| % Recovery | 95 to 110% | 89 to 105% | 92 to 96% | 88 to 99% |

With regards to the ICP-MS analysis, the Target elements were chosen based on USP<232> and ICH Q3D guidelines. For both Product Dish long-term storage leachable and Hold Dish sub-leachable samples, all tested elements were found below their respective limits with most results being either non-detected (ND) or below LOQ (<LOQ; Table 9. For an impurity to be non-detected, the response in the sample is less than or equal to the response in the control.

TABLE 9
ICP-MS Limit of Quantitation per Element—Product
Dish/Hold Dish Leachables Studies

| Element | LOQ (ppb) |
|---|---|
| Cd, Pb, As, Hg, Co, V, Ni, Se, Li, Sb, Mo, and | 0.1 |
| Ba, Cu, Sn, Cr, Al, B, Ti, and Mn | 0.3 |

The PDE limits of target elements were converted to the concentration limits using 30 tissue per day, one tissue per Dish (Product Dish or Hold Dish), the volume of solvent per Dish, and the dilution factor (DF) in the calculation. This conservative approach is the same as the approach used to calculate AET for organic leachables in this study. Examples are provided in the footnote of the tables below.

The concentration limits of elements are different between the Hold Dish leachables study and the Product Dish long-term storage leachables study due to difference in the solvent volumes. In the long term storage leachables study, the volume of CPS is 3 mL per Product Dish. In the Hold Dish leachables study, a volume of 2.5 mL of the CPS and Hold Solution mixture per Hold Dish was used to calculate. This is the maximum volume of the CPS and Hold Solution mixture from the total 18 mL in the Hold Dish that would be contained in a single StrataGraft skin tissue applied to a patient.

Product Dish long term storage leachable results and respective limits per element are provided in Table 10 followed by Hold Dish leachables results and respective limits per element as provided in Table 11. All results were below their elements respective limits.

Aluminum was detected in the T=0 Product Dish leachate control sample at 0.54 ppb and 0.87 ppb with the latter being in the Water Control sample. WHO has a recommendation not to exceed 1.8 mg/day for an adult from drinking water. As such, the ppb level of aluminum is negligible, does not pose a risk to patients. Lithium (Li) and lead (Pb) were detected at 0.14 and 0.11 ppb respectively, which were well below their respective limits. Nickel was detected at 0.20 ppb in the Hold Dish leachables study, which was below its respective limit.

TABLE 10
Results of the Product Dish Leachables Study (Solvent = CPS)

| | | Parenterals Limit | | | Result (ppb) | |
|---|---|---|---|---|---|---|
| | | | | Target analyte | T = 0 | T = 0 |
| | | PDE | Concentration | concentration | CPS | Water |
| Element | Class | (μg/day)¹ | (μg/mL)² | (ng/ml or | control | control |

Group α

| | | | | | | |
|---|---|---|---|---|---|---|
| Cd | 1 | 2 | 0.022 | 0.11 | <LOQ | <LOQ |
| Pb | 1 | 5 | 0.055 | 0.28 | 0.11 | 0.12 |
| As | 1 | 15 | 0.167 | 0.84 | <LOQ | 0.14 |
| Hg | 1 | 3 | 0.033 | 0.17 | <LOQ | <LOQ |
| Co | 2A | 5 | 0.056 | 0.28 | <LOQ | <LOQ |
| V | 2A | 10 | 0.11 | 0.56 | <LOQ | 0.13 |
| Ni | 2A | 20 | 0.22 | 1.11 | <LOQ | 0.11 |
| Se | 2B | 80 | 0.89 | 4.45 | ND | <LOQ |

Group β

| | | | | | | |
|---|---|---|---|---|---|---|
| Li | 3 | 250 | 2.78 | 0.35 | 0.14 | <LOQ |
| Sb | 3 | 90 | 1 | 0.13 | <LOQ | <LOQ |
| Ba | 3 | 700 | 7.78 | 0.97 | <LOQ | <LOQ |
| Mo | 3 | 1500 | 16.7 | 2.08 | <LOQ | <LOQ |
| Cu | 3 | 300 | 3.33 | 0.42 | <LOQ | <LOQ |
| Sn | 3 | 600 | 6.67 | 0.83 | ND | <LOQ |
| Cr | 3 | 1100 | 12.2 | 1.53 | <LOQ | <LOQ |

Other

| | | | | | |
|---|---|---|---|---|---|
| B | N/A | N/A | N/A | <LOQ | <LOQ |
| Al | N/A | N/A | N/A | 0.54 | 0.87 |
| Ti | N/A | N/A | N/A | <LOQ | <LOQ |
| Mn | N/A | N/A | N/A | <LOQ | <LOQ |
| W | N/A | N/A | N/A | <LOQ | <LOQ |

LOQ = limit of quantitation; N/A = not applicable; ND = not detected.
¹Permitted Daily Exposure (PDE) limits for parenteral products based on ICHQ3D guidelines.
²Thirty tissues per day and 3 mL Cryopreservative solution (CPS) per Product Dish was applied to determine each elemental impurity concentration limit in μg/mL (ppm). Ex. Cd: 2 μg/day X day/30 tissues X tissue/3 mL = 0.022 μg/mL
³At sample dilution of 1:200 (Group α elements) or 1:8000 (Group β elements)
Thirty tissues per day and 3 mL CPS solution per Product Dish, and Dilution Factor were applied to determine each element impurity concentration limit in ng/mL. Cd example: 2 μg/day X day/30 tissues X tissue/3 mL × 1/200 × 1000 (ng) = 0.11 ng/mL (ppb)

TABLE 11
Hold Dish Leachables Study, Solvent = Hold Solution +
CPS: Limits and Results

| | | Parenterals Limit | | | Result |
|---|---|---|---|---|---|
| | | | | Target analyte | (ppb)⁴ |
| | | PDE | Concentration | concentration | T = 4 h, |
| Element | Class | (μg/day)¹ | (μg/mL)² | (ng/ml or ppb)³ | 37° C. |

Group α

| | | | | | |
|---|---|---|---|---|---|
| Cd | 1 | 2 | 0.027 | 0.13 | ND |
| Pb | 1 | 5 | 0.067 | 0.33 | ND |
| As | 1 | 15 | 0.200 | 1.00 | ND |
| Hg | 1 | 3 | 0.040 | 0.20 | ND |
| Co | 2A | 5 | 0.067 | 0.33 | ND |
| V | 2A | 10 | 0.133 | 0.67 | ND |
| Ni | 2A | 20 | 0.267 | 1.33 | 0.20 |
| Se | 2B | 80 | 1.07 | 5.33 | ND |

Group β

| | | | | | |
|---|---|---|---|---|---|
| Li | 3 | 250 | 3.33 | 0.42 | ND |
| Sb | 3 | 90 | 1.20 | 0.15 | <LOQ |
| Ba | 3 | 700 | 9.33 | 1.17 | <LOQ |
| Mo | 3 | 1500 | 20.0 | 2.50 | ND |
| Cu | 3 | 300 | 4.0 | 0.50 | ND |
| Sn | 3 | 600 | 8.0 | 1.00 | <LOQ |
| Cr | 3 | 1100 | 14.7 | 1.83 | ND |

Other

| | | | | |
|---|---|---|---|---|
| B | N/A | N/A | N/A | <LOQ |
| Al | N/A | N/A | N/A | 0.54 |
| Ti | N/A | N/A | N/A | <LOQ |
| Mn | N/A | N/A | N/A | <LOQ |
| W | N/A | N/A | N/A | ND |

LOQ = limit of quantitation; N/A = not applicable; ND = not detected.
¹Permitted Daily Exposure (PDE) limits for parenteral products based on ICHQ3D guidelines.
²Thirty tissues per day and 2.5 mL of a mixture of CPS and Hold Solution per Hold dish was applied to determine each elemental impurity concentration limit in μg/mL (ppm). Ex. Cd: 2 μg/day × day/30 tissues × tissue/2.5 mL = 0.027 μg/mL
³At sample dilution of 1:200 (Group α elements) or 1:8000 (Group β elements)
Thirty tissues per day and 2.5 mL of a mixture of CPS and Hold Solution per Hold dish, and Dilution Factor were applied to determine each element impurity concentration limit in ng/ml. Cd example: 2 μg/day × day/30 tissues tissue/2.5 mL × 1/200 × 1000 (ng) = 0.13 ng/ml (ppb).
⁴The detected amount in the control samples was subtracted from the test samples

In elemental analysis for T=0M stability study, Pb, As, V, Ni, and Al were detected. In cases where the response of an element in the sample was less than or equal to its response in the control, the result reported as non-detected (ND) since the result was not differentiated from the control and below LOQ (<LOQ) for elements detected in samples below the Limit of Quantitation.

In elemental analysis, Ni and Al were detected. In cases where the response of an element in the sample was less than or equal to its response in the control, the result reported as non-detected (ND) since the result was not differentiated from the control and below LOQ (<LOQ) for elements detected in samples below the Limit of Quantitation. Leachables will be monitored at all remaining time points for the Product Dish study and results will be pooled with the Hold Dish leachables results provided herein.

1. A composition of allogeneic cultured keratinocytes for topical use, the composition comprising an allogeneic cellularized scaffold, wherein the scaffold further comprises dermal fibroblasts and murine collagen.

2. The composition of claim 1, wherein the keratinocytes and dermal fibroblasts are karyotypically stable.

3. The composition of claim 1, wherein the keratinocytes and dermal fibroblasts do not exhibit anchorage-independent growth.

4. The composition of claim 1, wherein the composition comprises no detectable pathogens.

5. The composition of claim 1, wherein the composition secretes human growth factors and cytokines.

6. The composition of claim 1, wherein the composition induces no immune rejection within 12 months of topical use.

7. The composition of claim 1, wherein the dermal fibroblasts are human dermal fibroblasts.

8. The composition of claim 1, wherein the composition further comprises human extracellular matrix proteins.

9. The composition of claim 1, wherein the composition further comprises glycerin.

10. The composition of claim 1, wherein the keratinocytes are cultured for 43 passages.

11. The composition of claim 1, wherein the dermal fibroblasts are cultured for 6 passages.

12. The composition of claim 1, wherein the composition is derived from human keratinocyte and fibroblast cell banks that contain no detectable pathogens.

13. The composition of claim 1, wherein the cellularized scaffold construct is rectangular in shape or trimmed to the shape of a wound bed.

14. The composition of claim 1, wherein the cellularized scaffold construct is for application to a single patient only.

15. The composition of claim 1, wherein the composition is prescribed for the treatment of adults with thermal burns in unit dosages, and wherein the unit dosage is an approximately 100 cm² rectangle.

16. The composition of claim 15, wherein the rectangular unit dosage is approximately 8 cm by 12.5 cm.

17. The composition of claim 1, wherein the composition is prescribed for the treatment of adults with thermal deep partial-thickness burns in unit dosages, and wherein the unit dosage is an approximately 100 cm² rectangle.

18. The composition of claim 17, wherein the rectangular unit dosage is approximately 8 cm by 12.5 cm.

19. The composition of claim 1, wherein the composition provides durable wound closure at a thermal burn treatment site at 3 months without autograft placement.

20. The composition of claim 1, wherein the composition is a xenotransplantation product.

21. The composition of claim 1, wherein mouse cells are not used in the manufacture of the composition.

22. The composition of claim 1, further comprising a packaging material gamma irradiated at a target minimum dose of 30.0 kGy.

23. The composition of claim 1, wherein the composition has been tested using USP <87> (in vitro biological reactivity) and were found to exhibit no cytotoxicity.

24. The composition of claim 1, wherein the composition has been tested using USP <88> (in vivo biological reactivity) and meet Class VI standards.

25. The composition of claim 1, wherein the composition is retained at 37±2° C. in a humidified, 5% CO₂ incubator for at least 25 days.

26. The composition of claim 1, further comprising a packaging layer having an oxygen transmission less than 0.003 cc/100 in²/24 hours.

27. The composition of claim 1, further comprising a packaging layer having a moisture permeation of less than 0.0065 g/100 cm²/24 hours.

28. The composition of claim 1, further comprising a 30 gauge layer aluminum foil pouch.

29. The composition of claim 1, further comprising an 18 pt cardboard carton comprising an opacity percentage of 99.74%.

30. The composition of claim 1, wherein the composition is stored inside a foil pouch until preparation for clinical use.

31. The composition of claim 22, wherein the foil pouch does not contact the composition during packaging, shipping, or clinical use.

32. The composition of claim 22, wherein the composition is cryopreserved within the foil until preparation for clinical use.

33. The composition of claim 1, wherein the composition is substantially free of antimicrobial compositions selected from the group consisting of mafenide acetate, silver-containing antimicrobials, chlorhexidine solution, and combinations thereof.

34. The composition of claim 33, wherein the composition has improved efficacy treating adult thermal burns in the absence of antimicrobial compositions.

35. The composition of claim 2, wherein a single subcutaneous injection of the keratinocytes into immunodeficient mice does not result in tumor formation by 23 weeks post-injection.

36. The composition of claim 2, wherein a topical application of the composition on full-thickness excisional wounds in immunodeficient mice does not result in tumor formation by 20 weeks post-dose.

37. The composition of claim 2, wherein no evidence of local or systemic toxicity is observed in immunocompromised athymic nude mice contacted with the composition.

38.-95. (canceled)
